(19) United States
(12) Patent Application Publication
(10) Pub. No.: US 2026/0311841 A1
(43) Pub. Date: Oct. 8, 2026

(54) OPPORTUNITY ANALYSES FOR RADIOTHERAPY SYSTEMS

(71) Applicant: Elekta, Inc., Atlanta, GA (US)

(72) Inventor: Benjamin Edward Nelms, Merrimac, WI (US)

(21) Appl. No.: 19/481,625

(22) PCT Filed: Jan. 29, 2024
(86) PCT No.: PCT/US2024/013316

§ 371 Date: Nov. 5, 2025

Related U.S. Application Data
(60) Provisional application No. 63/501,000, filed on May 9, 2023.

(51) Int. Cl. G16H 20/40; G16H 30/40; G16H 50/20

(52) U.S. Cl. CPC G16H 20/40; G16H 30/40; G16H 50/20

(57) ABSTRACT

Dose-sparing opportunities may be determined using what are referred to as opportunity dose-volume histogram (ODVH), where an ODVH is modelled based on an anatomy of the patient in an imaging or radiotherapy treatment session and depends to some degree on the treatment modality (e.g., energy of the x-rays or gamma-rays, degrees of freedom in the external beam arrangements, and the ideal target dose patterns).

### CLAIM OF PRIORITY

This application claims the benefit of priority of U.S. Provisional Patent Application Ser. No. 63/501,000, titled “PERSONALIZED PRESCRIPTIONS AND OPPORTUNITY ANALYSES FOR RADIOTHERAPY SYSTEMS” to Benjamin Edward Nelms, filed on May 9, 2023, the entire contents of which being incorporated herein by reference.

### TECHNICAL FIELD

Embodiments of the present disclosure pertain generally to patient-specific evaluation and optimization of treatment plans in radiotherapy treatment.

### BACKGROUND

Radiation therapy (or “radiotherapy”) may be used to treat cancers or other ailments in mammalian (e.g., human and animal) tissue. One such radiotherapy technique involves irradiation with a Gamma Knife®, whereby a patient is irradiated by a large number of low-intensity gamma ray beams that converge with high intensity and high precision at a target (e.g., a tumor). In another embodiment, radiotherapy is provided using a linear accelerator, whereby a tumor is irradiated by high-energy beams (e.g., electrons, protons, ions, high-energy photons, and the like). The placement and dose of the radiation beam must be accurately controlled to ensure the tumor receives the prescribed radiation, and the placement of the beams should be such as to minimize damage to the surrounding healthy tissue, often called the organ(s) at risk (OARs). Radiation is termed “prescribed” because a physician orders a predefined amount of radiation to be deposited in the target volumes as well as limits to the doses received (or volumes receiving certain dose levels) of the surrounding organs, similar to a prescription for medicine. Generally, ionizing radiation in the form of a collimated beam is directed from an external radiation source toward a patient but the radiation may also come from radioactive seeds located in the tumor.

A specified or selectable beam energy may be used, such as for delivering a diagnostic energy level range or a therapeutic energy level range. Modulation of a radiation beam may be provided by one or more attenuators or collimators (e.g., a multi-leaf collimator (MLC)). The intensity and shape of the radiation beam may be adjusted by collimation to avoid damaging healthy tissue (e.g., OARs) adjacent to the targeted tissue by conforming the projected beam to a profile of the targeted tissue.

The treatment planning procedure may include using a three-dimensional (3D) image of the patient to identify a target region (e.g., the tumor) and to identify critical organs near the tumor. Creation of a treatment plan may be a time-consuming process where a planner tries to comply with various treatment objectives or constraints (e.g., dose-volume histogram (DVH), overlap volume histogram (OVH)), taking into account their individual importance (e.g., weighting) in order to produce a treatment plan that is clinically acceptable. This task may be a time-consuming trial-and-error process that is complicated by the various OARs because as the number of OARs increases (e.g., up to thirteen for a head-and-neck treatment), so does the complexity of the process. OARs distant from a tumor may be easily spared from radiation, while OARs close to or overlapping a target tumor may be difficult to spare.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example but not by way of limitation, various embodiments discussed in the present document.

FIG. 1 illustrates an example of a radiotherapy system for providing radiation therapy to a patient.

FIGS. 2A and 2B are a flow diagram representing a radiation therapy workflow and the places available for the opportunity analysis techniques of this disclosure.

FIG. 3 is a graph illustrating an example of using patient-specific opportunities to personalize prescriptions for specific patient anatomies by sparing a healthy organic beyond generic protocol goals.

FIG. 4 is a graph illustrating another example of using patient-specific opportunities to personalize prescriptions, this time quantifying in advance any generic protocol goals that may be physically impossible.

FIG. 5A is a graph depicting an example of an achieved/planned DVH compared to a patient-specific anatomy-based Opportunity DVH (ODVH) curve.

FIG. 5B is a graph depicting an example of the opportunities for sparing an OAR comparing one instance of a patient's anatomy to another instance (e.g., one treatment fraction versus another, where the internal organs and volumes have changed in shape and/or location). FIG. 6A depicts an example an opportunity scorecard, in accordance with this disclosure.

FIG. 6B shows examples of opportunity scorecard results viewed in conjunction with generic protocol goals.

FIG. 7 is a chart illustrating the relationship between steps, anatomy, and treatment plans (both initial and per-fraction/adaptive).

FIG. 8 is a flow diagram illustrating an adaptive radiotherapy planning process utilizing the adaptive opportunity assistance techniques of this disclosure.

FIG. 9 is a conceptual diagram illustrating an example of an adaptive opportunity assistant for a treatment fraction “i”.

FIG. 10 depicts a graph illustrating an example of analyzing a radiotherapy dose-sparing opportunity for a subject using the “opportunity cost” methods.

FIG. 11 is a flow diagram of an example of a computer-implemented method 1100 of analyzing a radiotherapy dose-sparing opportunity for a subject.

FIG. 12 illustrates a block diagram of an embodiment of a machine on which one or more of the methods as discussed herein may be implemented.

FIG. 13 is a flow diagram illustrating an example of an adaptive radiotherapy planning process utilizing opportunity-assisted decision support techniques in accordance with this disclosure.

FIG. 14 depicts an example of a scorecard depicting an aggregation of opportunity costs over multiple OARs for plan evaluation, in accordance with this disclosure.

FIG. 15 is a flow diagram of an example of a computer-implemented method of evaluating a radiotherapy plan for a patient.

### DETAILED DESCRIPTION

In the following detailed description, reference is made to the accompanying drawings which form a part hereof, and which is shown by way of illustration-specific embodiments in which the present disclosure may be practiced. These embodiments, which are also referred to herein as “examples,” are described in sufficient detail to enable those skilled in the art to practice the disclosure, and it is to be understood that the embodiments may be combined, or that other embodiments may be utilized, and that structural, logical, and electrical changes may be made without departing from the scope of the present disclosure. The following detailed description is, therefore, not be taken in a limiting sense, and the scope of the present disclosure is defined by the appended claims and their equivalents.

Traditionally, for each patient, an initial treatment plan may be generated in an “offline” manner. The treatment plan may be developed well before radiation therapy is delivered, such as using one or more medical imaging techniques. Imaging information may include, for example, images from X-rays, computed tomography (CT), nuclear magnetic resonance (MR), positron emission tomography (PET), single-photon emission computed tomography (SPECT), or ultrasound. A healthcare provider, such as a physician, may use 3D imaging information indicative of the patient's anatomy to identify one or more target tumors along with the OARs near the tumor(s). The healthcare provider may delineate the target tumor that is to receive a prescribed radiation dose using a manual technique, and the healthcare provider may similarly delineate nearby tissue, such as organs, at risk of damage from the radiation treatment. Alternatively, or additionally, an automated tool (e.g., ABAS provided by Elekta AB, Sweden) may be used to assist in identifying or delineating the target tumor and organs at risk. A radiation therapy treatment plan (“treatment plan”) may then be created using an optimization technique based on clinical and dosimetric objectives and constraints (e.g., the maximum, minimum, and fraction of dose of radiation to a fraction of the tumor volume (“95% of the target shall receive no less than 100% of prescribed dose”), and like measures for the critical organs). The optimized plan is comprised of numerical parameters that specify the direction (static or dynamic, i.e., rotating), cross-sectional shape (static or dynamic, i.e., intensity-modulated), and photon fluence (constant or dynamic, i.e., varying as a function of time) of each radiation beam.

The treatment plan may then be later executed by positioning the patient in the planned treatment position, which is registered to the treatment machine, and then delivering the prescribed radiation therapy dose directed by the optimized plan parameters. The radiation therapy treatment plan may include dose “fractioning,” whereby a sequence of radiation treatments is provided over a period of time at a predetermined frequency (e.g., 30-45 fractions delivered one/day, five days a week for conventional fractionation; or, a smaller number of fractions, typically from one to five, for a technique known as hypofractionation and is used for tumors with cells that might be killed more easily in higher doses and fewer fractions). Each treatment fraction will deliver a portion of the predefined total prescribed dose. For each fraction, the position of the patient and the position of the target volumes and internal organs may change, and it is important to track and adapt to this, and sometimes reoptimize a new plan (called an “adaptive plan”) in real-time to ensure the target tumor and not healthy tissue is irradiated. This requires 3D imaging on the treatment machine, and rapid anatomical delineation and, if required and possible, the creation, approval, and delivery of an adapted fractional plan.

The present inventor has recognized that it is important to objectively and comprehensively measure the quality of a personalized treatment plan. The present inventor has recognized that including the target coverage as well as the sparing of the OARs is required, and the importance of each should be accounted for. Desirably, the quality measures analyze the entire clinically-relevant dose- and partial-volume ranges for each OAR and are not limited to discrete points on a DVH. Also, the present inventor has recognized that it would be beneficial for the quality measures to be “personalized” for each individual patient's unique anatomy, such as by gauging quality versus patient-specific achievable limits (“opportunities”) or other personalized, anatomy-specific baselines. In this way, patient-specific analyses of achievements versus opportunities create personalized quality metrics to use—in addition or instead of—the generic objectives that are typically used for all patients across a group of similar patients and prescriptions.

Furthermore, since most patients receive more than one fraction of radiation as part of a course of therapy, the anatomy may change (deform) between these fractions. As such, each individual fraction has unique challenges related to variation in anatomy, and the intended plan quality should be re-evaluated for that fraction's anatomy and adapted to create a “fractional plan” or adaptive plan that maximizes the quality for each particular anatomy instance. It is important to create a new (or find an existing) adapted plan as quickly as possible, because for online adaptive planning, the patient remains immobilized on the treatment table during the process. State-of-the-art radiation therapy analysis uses a tool called the dose-volume histogram (DVH). A DVH is a graph that reduces 3D data (anatomy volumes and 3D radiation dose grids) to quantify the distribution of radiation doses delivered inside a particular volume or organ of interest in radiation therapy. A typical DVH shows the percentage of the volume or organ receiving a certain radiation dose. The horizontal axis represents the dose, usually measured in units of gray (Gy), and the vertical axis represents the percentage of the volume receiving that dose. The DVH makes it easier for a clinician to examine the dose coverage or sparing over a volume, without having to visualize isodose lines and/or dose clouds overlaying a multitude of sample 2D slices through a patient (e.g., dose visualized over a long series of axial CT images). In general, clinicians gauge the coverage of dose to target volumes and the sparing of dose to critical organs at risk (OAR) to which they want to avoid or minimize collateral damage. These clinicians (e.g., dosimetrists, physicians, or radiation therapists) often use protocol goals, or “generic goals” (i.e., those that would be used across all patients in the same cohort) that are based on DVH curves to define the radiation-sparing objectives for important OARs. The protocol goal for each OAR is often defined using one or more points on DVH curve. To achieve a DVH goal for an OAR to be spared, the DVH curve should stay under the defined objective point or points on the DVH.

The generic protocol goals are usually derived based on large cohort outcomes studies, and though it would be ideal to personalize them to patient-specific anatomy and/or biology, this is rarely done today. The present inventor has recognized that there may be situations in which the protocol goal may be improved upon in ways very specific to each unique patient anatomy, which leads to patient-specific radiotherapy dose-sparing opportunities. For example, if a patient's anatomy changed since a previous treatment fraction, that change may result in amplified dose-sparing opportunities for the OAR compared to the initial (pre-treatment) radiation plan. The present inventor has recognized that these dose-sparing opportunities may be determined using what is referred to in this disclosure as an opportunity dose-volume histogram (ODVH), where an ODVH is modelled based on an anatomy of the patient in an imaging or radiotherapy treatment session and depends to some degree on the treatment modality (e.g., the energy of the x-rays or gamma-rays, degrees of freedom in the external beam arrangements, and the ideal target dose patterns).

An ODVH is an estimate of what may be achieved by external beam photon treatments for any specific patient. There are many ways by which an ODVH may be generated, including but not excluded to actual optimizations by the planning system (computed in the background, and automated as soon as anatomical volumes are defined), A.I.-based deep learning methods based on a curated population of prior treatment plans, or model-based fast estimates (which are useful for online adaptive where time is of the essence with the patient present and in the treatment position). Using any computational method, the goal is that a lower band represents an achievable dose-sparing situation for a particular OAR that has full sparing priority, and an upper band represents what should be easily achievable even when surrounding OARs are also given sparing priority. It is desirable that the ODVH use cases defined in this application to have ODVH guidelines that are realistic, i.e., achievable, in the clinical sense. This is in contrast to fictitious dose grids that are explicitly designed to model unachievable limits based on basic principles of energy transfer physics, such as described in U.S. Pat. No. 9,463,336. Such unachievable limits are interesting in certain contexts, but not if the purposes include providing dose goals in advance of optimization, or quantifying the quality of real-world plans, because they represent overly aggressive sparing scenarios. The techniques described in U.S. Pat. No. 9,463,336 also limit the target dose to assumed 100% coverage and uniform pattern, neither of which is realistic in the clinical sense.

This disclosure describes the use of Opportunity-Derived Quality Metrics. Using various techniques described below, an input DVH (e.g., ODVH) may be used as a reference against which comparison curves (e.g., planned DVHs from various modalities or methods, or ODVH from different anatomy arrangements) may be analyzed to produce objective quality metrics that span continuous dose ranges (or the whole dose range) for more comprehensive analysis of the OAR dose over its whole volume, and these per-OAR quality metrics may be aggregated across all OARs to generate comprehensive quality metric(s).

This disclosure also describes the use of Opportunity-Assisted Adaptive Planning. Using various techniques described below, two ODVHs may be determined from two different imaging or radiotherapy treatment sessions. Then, an updated radiotherapy dose-sparing opportunity for any critical OAR may be determined based on a difference between the two ODVHs. An output that represents the radiotherapy dose-sparing opportunity for the OAR may then be generated and displayed to a user, and the ODVH differences could be used to guide the plan/dose optimization for the new anatomy (i.e., the new treatment fraction). In addition, a prior opportunity dose (derived from a prior imaging session's anatomy) may be analyzed versus the current opportunity dose (derived from the new anatomy) to see if that existing plan, optimized to the prior session, would be acceptable for today's session without having to take the time to optimize yet another plan from scratch. Both methods of using opportunity-guided assistance lead to an adapted treatment plan, one that is either (a) selected from an inventory of pre-calculated plans based on an opportunity match, or (b) re-optimized with ODVH-guided shifts in goals.

In other words, based on the displayed output, the user may be able to quickly decide that because clinically significant dose-sparing opportunities are available, it is worth the time and effort, and the potential additional discomfort to the subject (e.g., if they are already immobilized and waiting for that fractional treatment) to optimize a new plan specific to the current session's anatomy. However, if the displayed output indicates that any dose-sparing opportunities are not clinically significant or that there are no dose-sparing opportunities, then the user may proceed with the previously generated radiation therapy treatment plan and not take up valuable time trying to modify, e.g., adapt or optimize, the treatment plan. These techniques allow a user to quickly determine whether the additional time (which may be up to 10-20 minutes needed to create an adapted treatment plan) is justified based on the potential to improve plan quality weighted against the longer treatment time and thus potential discomfort to the subject.

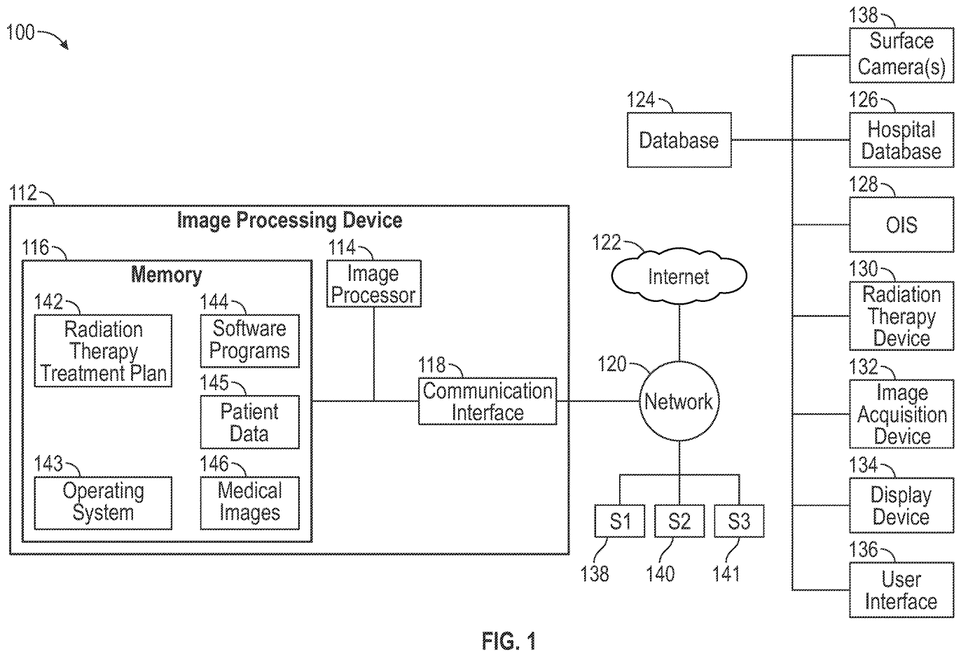

FIG. 1 illustrates an example of a radiotherapy system 100 for providing radiation therapy to a patient. The radiotherapy system 100 includes an image processing device 112. The image processing device 112 may be connected to a network 120. The network 120 may be connected to the Internet 122. The network 120 may connect the image processing device 112 with one or more of a database 124, a hospital database 126, an oncology information system (OIS) 128, a radiation therapy device 130, an image acquisition device 132, a display device 134, a user interface 136, and one or more surface cameras 138. Examples of surface cameras 138 may include those manufactured by C-Rad, VisionRT, and Varian HumediQ. The surface camera(s) 138 (e.g., one or more 2D or 3D cameras) may be used to acquire real-time images of the surface of a patient's body (e.g., the patient's skin) while medical images are being acquired. For Gamma Knife® mask treatments, an IR camera registers movements of markers fixed on the nose of the patient. Because the surface imaging is taken at the same time as the medical imaging, the surface imaging may provide a more accurate definition of the location of the boundaries of the patient's body while the medical imaging was taken. The image processing device 112 may be configured to generate radiation therapy treatment plans 142 to be used by the radiation therapy device 130.

The image processing device 112 may include a memory device 116, an image processor 114, and a communication interface 118. The memory device 116 may store computer-executable instructions, such as an operating system 143, radiation therapy treatment plans 142 (e.g., original treatment plans, adapted treatment plans and the like), software programs 144 (e.g., artificial intelligence, deep learning, neural networks, radiotherapy treatment plan software), and any other computer-executable instructions to be executed by the image processor 114.

In one embodiment, the software programs 144 may convert medical images of one format (e.g., MRI) to another format (e.g., CT) by producing synthetic images, such as pseudo-CT images. For instance, the software programs 144 may include image processing programs to train a predictive model for converting a medical image 146 in one modality (e.g., an MRI image) into a synthetic image of a different modality (e.g., a pseudo-CT image); alternatively, the trained predictive model may convert a CT image into an MRI image.

In another embodiment, the software programs 144 may register the patient image (e.g., a CT image or an MR image) with that patient's dose distribution (also represented as an image) so that corresponding image voxels and dose voxels are associated appropriately by the network.

In yet another embodiment, the software programs 144 may substitute functions of the patient images or processed versions of the images that emphasize some aspect of the image information. Such functions might emphasize edges or differences in voxel textures, or any other structural aspect useful to neural network learning.

In another embodiment, the software programs 144 may substitute functions of the dose distribution that emphasize some aspect of the dose information. Such functions might emphasize steep gradients around the target or any other structural aspect useful to neural network learning. The memory device 116 may store data, including medical images 146, patient data 145, and other data required to create and implement a radiation therapy treatment plan 142.

In yet another embodiment, the software programs 144 may generate a structural estimate (e.g., a 3D model of the region of interest) using an iterative image reconstruction process. The structural estimate may be or include an X-ray attenuation map that represents a 3D model of a region of interest. The structural estimate may be used to estimate or simulate X-ray measurements to be compared with real X-ray measurements for updating the structural estimate. Specifically, the software programs 144 may access a current structural estimate of the region of interest and generate a first simulated X-ray measurement based on the current structural estimate of the region of interest.

A simulated X-ray measurement, as referred to herein, represents the expected output of an X-ray detector element when an X-ray source projects one or more X-ray beams through the region of interest towards the X-ray detector element. The simulated X-ray measurement may provide an expected image output that is to be received from the X-ray detector element.

The software programs 144 may receive a first real X-ray measurement from a CBCT system (or other CT imaging system, such as an enclosed gantry helical multi-slice CT with a curved detector or tomotherapy system) and generate an update to the current structural estimate of the region of interest as a function of the first simulated X-ray measurement and the first real X-ray measurement. A real X-ray measurement, as referred to herein, is an actual output that is received from a CBCT system (or other CT imaging system, such as an enclosed gantry helical multi-slice CT with a curved detector or tomotherapy system) that represents the amount of signal generated by X-rays in the detector along different directions, such as in an image form.

The update may be generated invariant on (independent of) the current structural estimate. The structural estimate may be used to control one or more radiotherapy treatment parameters by recalculating dose, adjusting one or more radiotherapy treatment machine parameters, or generating a display of the structural estimate on a graphical user interface.

In addition to the memory device 116 storing the software programs 144, it is contemplated that software programs 144 may be stored on a removable computer medium, such as a hard drive, a computer disk, a CD-ROM, a DVD, a HD, a Blu-Ray DVD, USB flash drive, a SD card, a memory stick, or any other suitable medium; and the software programs 144 when downloaded to image processing device 112 may be executed by image processor 114.

The processor 114 may be communicatively coupled to the memory device 116, and the processor 114 may be configured to execute computer-executable instructions stored thereon. The processor 114 may send or receive medical images 146 to memory device 116. For example, the processor 114 may receive medical images 146 from the image acquisition device 132 via the communication interface 118 and network 120 to be stored in memory device 116. The processor 114 may also send medical images 146 stored in memory device 116 via the communication interface 118 to the network 120 be either stored in database 124 or the hospital database 126.

Further, the processor 114 may utilize software programs 144 (e.g., a treatment planning software) along with the medical images 146 and patient data 145 to create the radiation therapy treatment plan 142. Medical images 146 may include information such as imaging data associated with a patient anatomical region, organ, or volume of interest segmentation data. Patient data 145 may include information such as (1) functional organ modeling data (e.g., serial versus parallel organs, appropriate dose response models, etc.); (2) radiation dosage data (e.g., DVH information); or (3) other clinical information about the patient and course of treatment (e.g., other surgeries, chemotherapy, previous radiotherapy, etc.).

In addition, the processor 114 may utilize software programs to generate intermediate data such as updated parameters to be used, for example, by a machine learning model, such as a neural network model; or generate intermediate 2D or 3D images, which may then subsequently be stored in memory device 116. The processor 114 may subsequently transmit the executable radiation therapy treatment plan 142 via the communication interface 118 to the network 120 to the radiation therapy device 130, where the radiation therapy plan will be used to treat a patient with radiation. In addition, the processor 114 may execute software programs 144 to implement functions such as image conversion, image segmentation, deep learning, neural networks, and artificial intelligence. For instance, the processor 114 may execute software programs 144 that train or contour a medical image; such software programs 144 when executed may train a boundary detector or utilize a shape dictionary.

The processor 114 may be a processing device, including one or more general-purpose processing devices such as a microprocessor, a central processing unit (CPU), a graphics processing unit (GPU), an accelerated processing unit (APU), or the like. More particularly, the processor 114 may be a complex instruction set computing (CISC) microprocessor, a reduced instruction set computing (RISC) microprocessor, a very long instruction Word (VLIW) microprocessor, a processor implementing other instruction sets, or processors implementing a combination of instruction sets. The processor 114 may also be implemented by one or more special-purpose processing devices such as an application-specific integrated circuit (ASIC), a field programmable gate array (FPGA), a digital signal processor (DSP), a System on a Chip (SoC), or the like. As would be appreciated by those skilled in the art, in some embodiments, the processor 114 may be a special-purpose processor rather than a general-purpose processor. The processor 114 may include one or more known processing devices, such as a microprocessor from the Pentium™, Core™, Xeon™, or Itanium® family manufactured by Intel™, the Turion™, Athlon™, Sempron™, Opteron™, FX™, Phenom™ family manufactured by AMD™, or any of various processors manufactured by Sun Microsystems. The processor 114 may also include graphical processing units such as a GPU from the GeForce®, Quadro®, Tesla® family manufactured by Nvidia™, GMA, Iris™ family manufactured by Intel™, or the Radeon™ family manufactured by AMD™. The processor 114 may also include accelerated processing units such as the Xeon Phi™ family manufactured by Intel™. The disclosed embodiments are not limited to any type of processor(s) otherwise configured to meet the computing demands of identifying, analyzing, maintaining, generating, and/or providing large amounts of data or manipulating such data to perform the methods disclosed herein. In addition, the term “processor” may include more than one processor (for example, a multi-core design or a plurality of processors each having a multi-core design). The processor 114 may execute sequences of computer program instructions, stored in memory device 116, to perform various operations, processes, methods that will be explained in greater detail below.

The memory device 116 may store medical images 146. In some embodiments, the medical images 146 may include one or more MRI images (e.g., 2D MRI, 3D MRI, 2D streaming MRI, four-dimensional (4D) MRI, 4D volumetric MRI, 4D cine MRI, etc.), functional MRI images (e.g., fMRI, DCE-MRI, diffusion MRI), CT images (e.g., 2D CT, cone beam CT, 3D CT, 4D CT), ultrasound images (e.g., 2D ultrasound, 3D ultrasound, 4D ultrasound), one or more projection images representing views of an anatomy depicted in the MRI, synthetic CT (pseudo-CT), and/or CT images at different angles of a gantry relative to a patient axis, PET images, X-ray images, fluoroscopic images, radiotherapy portal images, SPECT images, computer generated synthetic images (e.g., pseudo-CT images), aperture images, graphical aperture image representations of MLC leaf positions at different gantry angles, and the like. Further, the medical images 146 may also include medical image data, for instance, training images, ground truth images, contoured images, and dose images. In an embodiment, the medical images 146 may be received from the image acquisition device 132. Accordingly, image acquisition device 132 may include an MRI imaging device, a Megavolt (MV) imaging device, a CT imaging device, a CBCT imaging device, a PET imaging device, an ultrasound imaging device, a fluoroscopic device, a SPECT imaging device, an integrated linac and MRI imaging device, an integrated linac and CT imaging device, an integrated linac and CBCT imaging device, or other medical imaging devices for obtaining the medical images of the patient. The medical images 146 may be received and stored in any type of data or any type of format that the image processing device 112 may use to perform operations consistent with the disclosed embodiments.

MRI images may be acquired using various pulse sequences. Two of the basic pulse sequences include longitudinal (T1) and transverse (T2) relaxation time sequences that generate T1-weighted images and T2-weighted images, respectively. MRI contrast agents are a group of contrast media used to improve the visibility of internal body structures by increasing contrast between normal tissues and abnormal tissues in MRI. MRI contrast agents alter the T1 (longitudinal) and T2 (transverse) relaxation times of tissues and body cavities where they are present and, depending on the image weighting, may result in a higher or lower signal. T1 MRI contrast agents produce the “bright” contrast in a T1-weighted image, whereas T2 MRI contrast agents create “dark” contrast effects.

The memory device 116 may be a non-transitory computer-readable medium, such as a read-only memory (ROM), a phase-change random access memory (PRAM), a static random access memory (SRAM), a flash memory, a random access memory (RAM), a dynamic random access memory (DRAM) such as synchronous DRAM (SDRAM), an electrically erasable programmable read-only memory (EEPROM), a static memory (e.g., flash memory, flash disk, static random access memory) as well as other types of random access memories, a cache, a register, a CD-ROM, a DVD or other optical storage, a cassette tape, other magnetic storage device, or any other non-transitory medium that may be used to store information including image, data, or computer-executable instructions (e.g., stored in any format) capable of being accessed by the processor 114, or any other type of computer device. The computer program instructions may be accessed by the processor 114, read from the ROM, or any other suitable memory location, and loaded into the RAM for execution by the processor 114. For example, the memory device 116 may store one or more software applications. Software applications stored in the memory device 116 may include, for example, an operating system 143 for common computer systems as well as for software-controlled devices. Further, the memory device 116 may store an entire software application, or only a part of a software application, that is executable by the processor 114. For example, the memory device 116 may store one or more radiation therapy treatment plans 142.

The image processing device 112 may communicate with the network 120 via the communication interface 118, which may be communicatively coupled to the processor 114 and the memory device 116. The communication interface 118 may provide communication connections between the image processing device 112 and radiotherapy system 100 components (e.g., permitting the exchange of data with external devices). For instance, the communication interface 118 may, in some embodiments, have appropriate interfacing circuitry to connect to the user interface 136, which may be a hardware keyboard, a keypad, or a touch screen through which a user may input information into radiotherapy system 100.

Communication interface 118 may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as a WiFi adaptor), a telecommunication adaptor (e.g., 3G, 4G/LTE and the like), and the like. Communication interface 118 may include one or more digital and/or analog communication devices that permit image processing device 112 to communicate with other machines and devices, such as remotely located components, via the network 120.

The network 120 may provide the functionality of a local area network (LAN), a wireless network, a cloud computing environment (e.g., software as a service, platform as a service, infrastructure as a service, etc.), a client-server, a wide area network (WAN), and the like. For example, network 120 may be a LAN or a WAN that may include other systems S1 (138), S2 (140), and S3 (141). Systems S1, S2, and S3 may be identical to image processing device 112 or may be different systems. In some embodiments, one or more systems in network 120 may form a distributed computing/simulation environment that collaboratively performs the embodiments described herein. In some embodiments, one or more systems S1, S2, and S3 may include a CT scanner that obtains CT images (e.g., medical images 146). In addition, network 120 may be connected to Internet 122 to communicate with servers and clients that reside remotely on the internet.

Therefore, network 120 may allow data transmission between the image processing device 112 and a number of various other systems and devices, such as the OIS 128, the radiation therapy device 130, and the image acquisition device 132. Further, data generated by the OIS 128 and/or the image acquisition device 132 may be stored in the memory device 116, the database 124, and/or the hospital database 126. The data may be transmitted/received via network 120, through communication interface 118 in order to be accessed by the processor 114, as required.

The image processing device 112 may communicate with database 124 through network 120 to send/receive a plurality of various types of data stored on database 124. For example, database 124 may include machine data (control points) that includes information associated with a radiation therapy device 130, image acquisition device 132, or other machines relevant to radiotherapy. Machine data information may include control points, such as radiation beam size, arc placement, beam on and off time duration, machine parameters, segments, MLC configuration, gantry speed, MRI pulse sequence, and the like. Database 124 may be a storage device and may be equipped with appropriate database administration software programs. One skilled in the art would appreciate that database 124 may include a plurality of devices located either in a central or a distributed manner.

In some embodiments, database 124 may include a processor-readable storage medium. While the processor-readable storage medium in an embodiment may be a single medium, the term “processor-readable storage medium” should be taken to include a single medium or multiple media (e.g., a centralized or distributed database, and/or associated caches and servers) that store the one or more sets of computer-executable instructions or data. The term “processor-readable storage medium” shall also be taken to include any medium that is capable of storing or encoding a set of instructions for execution by a processor and that cause the processor to perform any one or more of the methodologies of the present disclosure. The term “processor-readable storage medium” shall accordingly be taken to include, but not be limited to, solid-state memories and optical and magnetic media. For example, the processor-readable storage medium may be one or more volatile, non-transitory, or non-volatile tangible computer-readable media.

Image processor 114 may communicate with database 124 to read images into memory device 116 or store images from memory device 116 to database 124. For example, the database 124 may be configured to store a plurality of images (e.g., 3D MRI, 4D MRI, 2D MRI slice images, CT images, 2D Fluoroscopy images, X-ray images, raw data from MR scans or CT scans, Digital Imaging and Communications in Medicine (DICOM) data, projection images, graphical aperture images, etc.) that the database 124 received from image acquisition device 132. Database 124 may store data to be used by the image processor 114 when executing software program 144 or when creating radiation therapy treatment plans 142. Database 124 may store the data produced by the trained machine learning mode, such as a neural network including the network parameters constituting the model learned by the network and the resulting estimated data. As referred to herein, “estimate” or “estimated” may be used interchangeably with “predict” or “predicted” and should be understood to have the same meaning. The image processing device 112 may receive the imaging data, such as a medical image 146 (e.g., 2D MRI slice images, CT images, 2D Fluoroscopy images, X-ray images, 3DMRI images, 4D MRI images, projection images, graphical aperture images, image contours, etc.) from the database 124, the radiation therapy device 130 (e.g., a linac or an MR-linac), and/or the image acquisition device 132 to generate a treatment plan 142. The radiation therapy device 130 may provide linac based treatments such as volumetric-modulated arc therapy (VMAT) or intensity modulated radiation therapy (IMRT), for example.

In an embodiment, the radiotherapy system 100 may include an image acquisition device 132 that may acquire medical images (e.g., MRI images, 3D MRI, 2D streaming MRI, 4D volumetric MRI, CT images, cone-Beam CT, PET images, functional MRI images (e.g., fMRI, DCE-MRI, and diffusion MRI), X-ray images, fluoroscopic image, ultrasound images, radiotherapy portal images, SPECT images, and the like) of the patient. Image acquisition device 132 may, for example, be an MRI imaging device, a CT imaging device, a PET imaging device, an ultrasound device, a fluoroscopic device, a SPECT imaging device, or any other suitable medical imaging device for obtaining one or more medical images of the patient. Images acquired by the image acquisition device 132 may be stored within database 124 as either imaging data and/or test data. By way of example, the images acquired by the image acquisition device 132 may be also stored by the image processing device 112 as medical images 146 in memory device 116.

In an embodiment, for example, the image acquisition device 132 may be integrated with the radiation therapy device 130 as a single apparatus (e.g., an MR-linac). Such an MR-linac may be used, for example, to determine a location of a target organ or a target tumor in the patient, so as to direct radiation therapy accurately according to the radiation therapy treatment plan 142 to a predetermined target.

The image acquisition device 132 may be configured to acquire one or more images, such as including spatial imaging data, of the patient's anatomy for a region of interest (e.g., a target organ, a target tumor, or both). Each image, typically a 2D image or slice, may include one or more parameters (e.g., a 2D slice thickness, an orientation, and a location, etc.). In an embodiment, the image acquisition device 132 may acquire a 2D slice in any orientation. For example, an orientation of the 2D slice may include a sagittal orientation, a coronal orientation, or an axial orientation. The processor 114 may adjust one or more parameters, such as the thickness and/or orientation of the 2D slice, to include the target organ and/or target tumor. In an embodiment, 2D slices may be determined from information such as a 3D MRI volume. Such 2D slices may be acquired by the image acquisition device 132 in “real-time” while a patient is undergoing radiation therapy treatment, for example, when using the radiation therapy device 130, with “real-time” meaning acquiring the data in at least milliseconds or less. The image acquisition device 132 may be configured to acquire 3D spatial imaging data.

The image processing device 112 may generate and store radiation therapy treatment plans 142 for one or more patients. The radiation therapy treatment plans 142 may provide information about a particular radiation dose to be applied to each patient. The radiation therapy treatment plans 142 may also include other radiotherapy information, such as control points including beam angles, gantry angles, beam intensity, dose-histogram-volume information, the number of radiation beams to be used during therapy, the dose per beam, and the like.

The image processor 114 may generate the radiation therapy treatment plan 142 by using software programs 144 such as treatment planning software (such as Leksell Gamma Plan® Monaco®, manufactured by Elekta, Sweden). In order to generate the radiation therapy treatment plans 142, the image processor 114 may communicate with the image acquisition device 132 (e.g., a CT device, an MRI device, a PET device, an X-ray device, an ultrasound device, etc.) to access images of the patient and to delineate a target, such as a tumor, to generate contours of the images. In some embodiments, the delineation of one or more OARs, such as healthy tissue surrounding the tumor or in close proximity to the tumor, may be required. Therefore, segmentation of the OAR may be performed when the OAR is close to the target tumor. In addition, if the target tumor is close to the OAR (e.g., prostate in near proximity to the bladder and rectum), then by segmenting the OAR from the tumor, the radiotherapy system 100 may study the dose distribution not only in the target but also in the OAR.

In order to delineate a target organ or a target tumor from the OAR, medical images, such as MRI images, CT images, PET images, fMRI images, X-ray images, ultrasound images, radiotherapy portal images, SPECT images, and the like, of the patient undergoing radiotherapy may be obtained non-invasively by the image acquisition device 132 to reveal the internal structure of a body part. Based on the information from the medical images, a 3D structure of the relevant anatomical portion may be obtained and used to generate a contour of the image. Contours of the image may include data overlaid on top of the image that delineates one or more structures of the anatomy. In some cases, the contours may be files associated with respective images that specify the coordinates or 2D or 3D locations of various structures of the anatomy depicted in the images.

In addition, during a treatment planning process, many parameters may be taken into consideration to achieve a balance between efficient treatment of the target tumor (e.g., such that the target tumor receives enough radiation dose for an effective therapy) and low irradiation of the OAR(s) (e.g., the OAR(s) receives as low a radiation dose as possible). Other parameters that may be considered include the location of the target organ and the target tumor, the location of the OAR, and the movement of the target in relation to the OAR. For example, the 3D structure may be obtained by contouring the target or contouring the OAR within each 2D layer or slice of an MRI or CT image and combining the contour of each 2D layer or slice. The contour may be generated manually (e.g., by a physician, dosimetrist, or healthcare worker using a program such as Leksell Gamma Plan® manufactured by Elekta) or automatically (e.g., using a program such as the Atlas-based auto-segmentation software, ABAS™, manufactured by Elekta). In certain embodiments, the 3D structure of a target tumor or an OAR may be generated automatically by the treatment planning software.

After the target tumor and the OAR(s) have been located and delineated, a dosimetrist, physician, or healthcare worker may determine a dose of radiation to be applied to the target tumor, as well as any maximum amounts of dose that may be received by the OAR proximate to the tumor (e.g., left and right parotid, optic nerves, eyes, lens, inner ears, spinal cord, brain stem, and the like). After the radiation dose is determined for each anatomical structure (e.g., target tumor, OAR), a process known as inverse planning may be performed to determine one or more treatment plan parameters that would achieve the desired radiation dose distribution. Examples of treatment plan parameters include volume delineation parameters (e.g., which define target volumes, contour sensitive structures, etc.), margins around the target tumor and OARs, beam angle selection, collimator settings, and beam-on times.

During the inverse-planning process, the physician may define dose constraint parameters that set bounds on how much radiation an OAR may receive (e.g., defining full dose to the tumor target and zero dose to any OAR; defining dose received by at least 95% of the target tumor volume; defining that the spinal cord, brain stem, and optic structures receive≤45 Gy, ≤55 Gy and <54 Gy, respectively). The result of inverse planning may constitute a radiation therapy treatment plan 142 that may be stored in memory device 116 or database 124. Some of these treatment parameters may be correlated. For example, tuning one parameter (e.g., weights for different objectives, such as increasing the dose to the target tumor) in an attempt to change the treatment plan may affect at least one other parameter, which in turn may result in the development of a different treatment plan. Thus, the image processing device 112 may generate a tailored radiation therapy treatment plan 142 having these parameters in order for the radiation therapy device 130 to provide radiotherapy treatment to the patient.

In addition, the radiotherapy system 100 may include a display device 134 and a user interface 136. The display device 134 may include one or more display screens that display medical images, interface information, treatment planning parameters (e.g., projection images, graphical aperture images, contours, dosages, beam angles, etc.) treatment plans, a target, localizing a target and/or tracking a target, or any related information to the user. The user interface 136 may be a keyboard, a keypad, a touch screen, or any type of device that a user may use to input information to radiotherapy system 100. Alternatively, the display device 134 and the user interface 136 may be integrated into a device such as a tablet computer (e.g., Apple iPad®, Lenovo Thinkpad®, Samsung Galaxy®, etc.).

Furthermore, any and all components of the radiotherapy system 100 may be implemented as a virtual machine (e.g., VMWare, Hyper-V, and the like). For instance, a virtual machine may be software that functions as hardware. Therefore, a virtual machine may include at least one or more virtual processors, one or more virtual memories, and one or more virtual communication interfaces that together function as hardware. For example, the image processing device 112, the OIS 128, and the image acquisition device 132 could be implemented as a virtual machine. Given the processing power, memory, and computational capability available, the entire radiotherapy system 100 could be implemented as a virtual machine.

As mentioned above, various techniques of this disclosure allow a clinician to quickly decide that because clinically significant sparing opportunities are available, it is worth the time and effort (which may take up to 10-20 minutes), and the potential additional discomfort to a patient, to modify, e.g., adapt or optimize, a previously generated radiation therapy treatment plan. Using these techniques allow a user to quickly determine whether the additional treatment time it takes to adapt a new treatment plan is justified, or if the initial plan (or any prior adapted plan) will suffice for the new 3D permutation of the patient's anatomy.

The industry standard plan evaluation tool called the dose-volume histogram (DVH) was introduced earlier, and it is worth going into more detail in order to set a foundation for the new techniques. The DVH is the most common object used to evaluate target coverage and organ-at-risk (OAR) dose-sparing in radiation treatment planning. A DVH is a data reduction object that is computed using inputs of a 3D radiation dose (calculated for a treatment plan) grid overlaid with 3D anatomical structures that were originally defined by drawing contours based on CT, MR, and/or other 3D imaging. A DVH computation involves interpolating the 3D dose grid into very finely resolved volume elements (“voxels”) and then assigning them over the anatomy to determine which voxels are part of any specific anatomical structure's volume (either inside, or on the surface).

For any given structure, the differential DVH is computed by binning the contained voxel's volume into small dose bins, e.g., a histogram computation with dose bins on the x-axis and volume on the y-axis. From the differential DVH, a cumulative DVH is then computed and is the preferred status quo that clinicians use. A cumulative DVH goes along the x-axis dose bins and assigns the total volume of the structure that receives greater than or equal to the dose of the bin. The cumulative DVH (usually referred to as just the DVH) helps a clinician (1) visualize an easy-to-interpret graph and also (2) extract specific values of interest. For example, a physician might want to know, “What volume of the left lung receives 20 Gy or more?” which would be a simple extraction of a point off the DVH curve, specifically the y-value of the curve at x=20 Gy. Or the physician might as, “What dose value covers 95% of the target volume?” which would be an extraction of the x-value at y=95% of the target volume. A DVH curve is a continuous function (only one or no y-values per x-value), computed empirically by modeling and counting 3D dose voxels, and stored as a data lookup table (LUT), not an equation.

This disclosure describes a new object called the Opportunity Dose-Volume Histogram (ODVH). An ODVH is calculated for any specific organ-at-risk for each unique permutation of a patient's unique anatomy, which includes target volumes and a collection of relevant organs-at-risk (OAR). An ODVH represents an estimate of the dose-volume range that is possible (lower edge) and likely (upper edge) for any OAR, given any specific dose prescription for targets, where dose is deposited from external photon radiation beams. The ODVH is estimated without any time-intensive inverse planning algorithms or modeling of actual radiation beams and relies only on the energy transfer characteristics of photons incident on living tissues. An actual radiation plan and its dose calculation (e.g., a calculated 3D dose grid for that plan) are not the inputs to an ODVH calculation; rather, what are required are the anatomic structures as well as the intended target volume(s) and the preferred dose coverage pattern(s). Also required are some practical considerations, such as if the clinicians intend to allow full beam geometries (i.e., allow non-coplanar beams) or limit to a single treatment couch angle (i.e., restrict to co-planar beam geometries). Other delivery inputs can be given, such as the photon source (e.g., GammaKnife radioactive Co-60 sources or external x-ray beams generated by linear accelerators) and the beam energy intended. With these inputs, statistical methods are used that accurately estimate dose-sparing possibilities (e.g., ranges) from first principles of external beam dose deposition (for the ODVH lower limit) as well as observed sparing results for multi-observer studies (for the ODVH upper limit model). The ODVH lower limit is an estimate of the DVH limits that could be achieved for this OAR if it is (1) the only priority and (2) is maximally spared given a collection of targets and target doses. The ODVH upper limit is an estimate of the doses/volumes that should be generally achievable for this OAR in the presence of other adjacent OARs that are also being spared, e.g., what DVH boundaries would be achieved by a certain percentile of human- or auto-generated plans. The ODVH, both graphically and in object form, is a “band” or “zone” in a dose-volume data table. Visually, an achieved plan DVH may be overlaid over an ODVH plot to see how an actual treatment plan and its calculated dose compares to the opportunity zone for any unique patient anatomical structure.

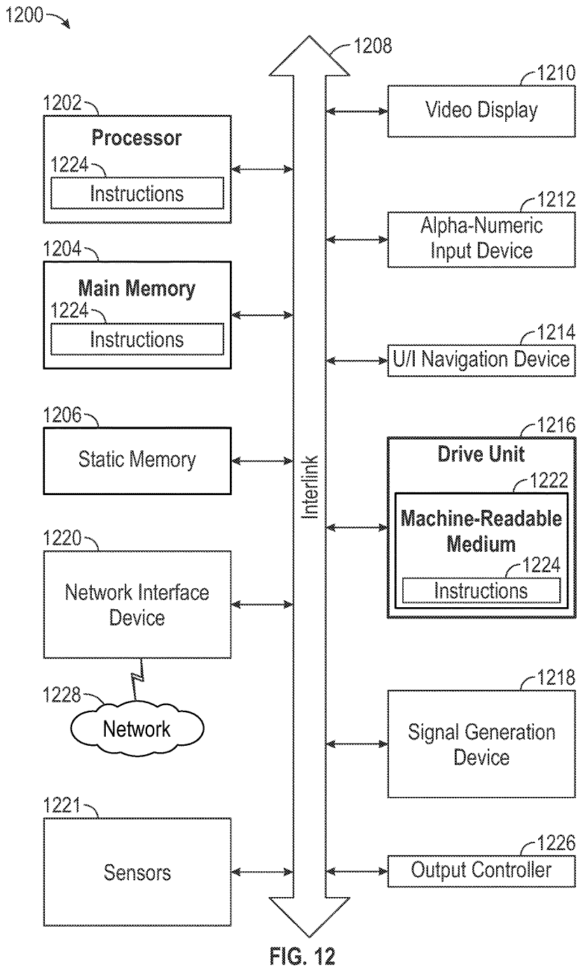

For any permutation of a human's anatomy modeled from 3D imaging, one or more processors, such as the image processing device 112 of FIG. 1 or the processor 1202 of FIG. 12, may automatically calculate an “Opportunity Grid” (OG) using these inputs: the anatomy model, the treatment technique (e.g., VMAT/IMRT as either non-coplanar beams or limited to coplanar arrangements, GammaKnife sources), and ideal target dose patterns (including intra-target variation) predefined by the user, such as a clinician. Each OG is a 3D matrix of vectors where the magnitude represents a quantified “resistance” to dose-sparing and the direction approximates the 3D sense of that resistance. A unique OG is created for any unique permutation of anatomy, and a series of OGs may be created across all such permutations, e.g., iterations of one human's 3D anatomy pretreatment and across treatment fractions. An OG is used in conjunction with the 3D anatomy models to calculate an Opportunity Dose-Volume Histogram (ODVH), where the lower limit is a fast estimate of what DVH may be achieved for the respective OAR if it is given full sparing priority. The upper limit estimates what is achievable as other competing OARs are included in the planning priorities.

The ODVH lower limit may also be calculated more robustly (though more time-intensively) by optimizing a non-clinical plan for each OAR, with that OAR maximally spared using the user-specified modality (e.g., VMAT or GammaKnife) and technique (non-coplanar beams versus coplanar beam constraint). An ODVH may also be calculated for each anatomy permutation “i” (Anatomyᵢ with OGᵢ, yielding ODVHᵢ) but also for that anatomy mingled with a different OG, e.g., the grid derived from a prior treatment's anatomy (Anatomyᵢ with OGⱼ).

ODVH may also inform the user about Personalized Prescription goals to supplement generic protocol goals that are not patient-specific. This may happen early in the workflow, as part of the creation of the intended treatment plan.

This disclosure also describes Opportunity Cost Metrics. Opportunity Cost (OC) metrics may be calculated from any pair of comparison and reference DVH curves. One example is if the reference is the ODVH lower limit and the comparison is the achieved DVH for an intended treatment plan that is to be evaluated for plan quality; the output in this case would be advanced measures of plan quality that are both patient-specific (i.e., personalized) but also comprehensive with respect to a clinically relevant dose range and not limited to discrete points extracted from a DVH.

Another example is if the reference represents a specific achievement or possibility for a specific modality or technique, while the comparison represents what is achieved or possible for an alternative modality or technique. One example of this application would be to quantify the potential advantage of heavy ion therapy, e.g., proton beams, for any patient compared to external beam photon therapy.

Another example is if the reference DVH is a fictitious, purposely-unachievable DVH (e.g., as described in U.S. Pat. No. 9,463,336), and the comparison is either an ODVH lower band or an achieved DVH from an intended treatment plan. In contrast to the techniques described in U.S. Pat. No. 9,463,336, the techniques of this disclosure do not generate a fictitious treatment dose matrix from the subset of radiation planning data with a modeling process, where the subset and the modeling process are selected so that the fictitious treatment dose matrix provides a quality of dose placement beyond that achievable with a physically realizable radiation therapy machine. The techniques of this disclosure also do not evaluate radiation planning information against a fictitious treatment dose matrix, where the evaluation indicates the probable impossibility of attaining at least a portion of the radiation plan goals. The ODVH, by contrast, is an accurate estimate of the dose sparing that can be achieved for any and all OARs, given a range of potential sparing priorities for each OAR.

Opportunity Cost metrics may also be calculated from any pair of ODVHs to quantify the relative opportunities for OAR dose-sparing (or expected difficulties in dose-sparing or target coverage) for two unique patient anatomy instances or two competing treatment techniques or prescriptions. In the case of two anatomy instances, the OC metrics may help find a “best match” of Anatomyᵢ versus any prior Anatomy_{(0 to i-1)} in a radiation dose sense for selection from an inventory of permutations of treatment plans, e.g., initial plan or prior treatment fractions' adapted plans.

The derivation of an Opportunity Cost removes the limits of typical metric checks that compare single points extracted from a curve (e.g., Volume of OAR receiving>30 Gy dosage, Dose to OAR covering 50% of the total volume, max dose to an OAR), and instead provides a comprehensive data-reduction technique that compares a comparison DVH to a reference DVH over a clinically-relevant dose range. A continuum of the curve's doses and volumes are analyzed and used in the computation, not limited to discrete points. An opportunity cost quantifies the differences between a comparison DVH (e.g., for an intended, planned treatment) and a reference DVH (e.g., the lower band of an ODVH), and may be computed to render an average volume difference, average dose difference, simple opportunity cost index (SOC index), or dose-weighted opportunity cost index (DOC index).

The opportunity analyses of this disclosure may be much faster than any process requiring an actual dose calculation and especially an inverse-planned dose optimization. They are also almost entirely anatomy-based (as well as physics-based) and not sensitive to specific treatment machine designs (e.g., multi-leaf collimator designs or other linear accelerator design specifics), with the exception of GammaKnife sources versus linear accelerator-generated external beam photons and allowed non-coplanar versus Coplanar beam arrangements. Thus, opportunity analyses make a stable, independent model for analyses rather than inviting undue variation in interpretation. For example, it's desirable to avoid an output that means one thing to a clinician using Machine A or Planning Software X, but may mean something different to a clinician using Machine B or Planning Software Y. Also, the OC metrics may provide clinicians comprehensive analytics reduced to simpler and fewer terms. In addition, patient-specific opportunity analyses may lead to higher quality plans (e.g., better normal tissue sparing) compared to those created using generic protocol objectives, for the initial plan but also for any/all treatment fractions where an adapted anatomy scan is acquired. As such, the techniques of this disclosure may improve efficiency and quality.

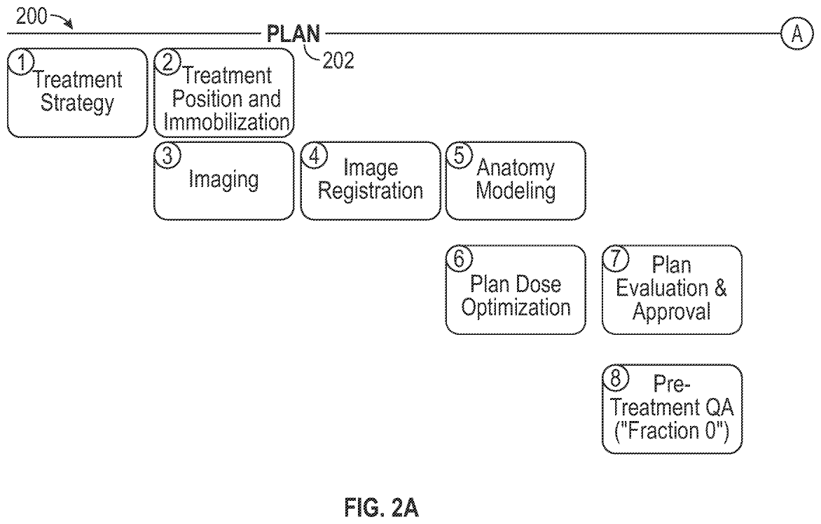

FIGS. 2A and 2B are a flow diagram representing a radiation therapy workflow and the places available for the opportunity analysis techniques of this disclosure. The radiation therapy workflow 200 includes three main portions: planning 202, treatment 204, and tracking 206. Opportunity analysis use cases exist for each of those three portions.

The planning portion 202 includes six steps, steps 1-6. Treatment strategy is step 1. Treatment position and immobilization is step 2. Imaging is step 3. Image registration is step 4. Anatomy modeling is step 5. Plan dose optimization is step 6. Plan evaluation and approval is step 7. Pretreatment quality assurance is step 8.

The treatment portion 204 includes 8 steps, steps A-H. Patient setup is step A. On-board imaging is step B. Deformable registration is step C. Anatomy modeling is step D. Fraction dose selection and re-optimization is step E. Fraction evaluation and approval is step F. Dose delivery is step G. Image registration and patient shifts is step H.

The tracking portion 206 includes 2 steps, steps 10 and 11. Dose accumulation is step 10. Monitor response and outcomes is step 11.

A first opportunity analysis use case is during the plan dose optimization step (step 6 in FIG. 2A) of the planning portion of the workflow, also referred to as a “personalized prescription” in this disclosure. The personalized prescription techniques may provide a patient-specific dose optimization wish list based on anatomic opportunities, such as a change in the positioning of patient anatomy between treatment fractions. The personalized prescription techniques may compute personalized prescriptions that capture patient-specific objectives to help optimize the plan beyond generic protocol goals. Similarly, the personalized prescription techniques may identify generic protocol objectives that may be challenging or impossible for a specific patient, which would be useful so that the user does not ask the inverse-planning engine to do beyond what is beyond the realistic opportunity for that particular patient.

A second opportunity analysis use case is during the plan evaluation and approval step (step 7 in FIG. 2A) of the planning portion of the workflow, also referred to as “plan evaluation” in this disclosure. Plan evaluation allows a clinician to assess plan quality against the patient-specific OAR-sparing opportunities to supplement assessments based on generic protocol goals. Plan evaluation may generate and display opportunity DVH (ODVH) curves against which to compare achieved DVH curves. In addition, plan evaluation may generate personalized scorecards (“Opportunity Scorecards”) to supplement generic protocol scorecards. Further, plan evaluation may calculate powerful new per-OAR quality/comparison metrics, called “Opportunity Cost” (OC) metrics, that are based on a pair of DVHs (e.g., comparing an OAR's achieved DVH for an intended plan to the ODVH for the same OAR) analyzed over a whole clinically-relevant range of dose/volumes (not limited to points on a curve).

A third opportunity analysis use case is during the fraction dose selection or re-optimization step and/or the fraction evaluation and approval step (steps 9E, 9F in FIG. 2B) of the treatment portion of the workflow, also referred to as an “adaptive opportunity assistant” in this disclosure. The adaptive opportunity assistant may provide a quantified and rapid decision support for adaptive radiation therapy. The adaptive opportunity assistant may provide rapid assessment of the sparing opportunity for fraction i versus the initial plan and versus prior fraction(s). In addition, the adaptive opportunity assistant may provide rapid analysis of the initial plan and over the library of prior fractions' adaptive plans to look for the best matches, allowing a clinician to select a prior plan for use rather than spend time adapting a current plan. Further, if a new adaptive plan is warranted, the adaptive opportunity assistant may help in understanding this fraction's unique opportunities (e.g., a personalized prescription, as described earlier, but for that specific treatment fraction's anatomy rather than the initial plan prior to treatment).

A fourth opportunity analysis use case is during the monitor response and outcomes step (step 11 in FIG. 2B) of the tracking portion of the workflow, also referred to as “outcomes analysis” in this disclosure. Outcomes analysis may use data and outcomes to mine for new metrics that are more predictive of outcome. Outcomes analysis may track the patient response (e.g., measures of normal function of OARs or toxicity to OARs) versus the opportunity-based metrics and mine for predictive power. Further, outcomes analysis may provide metrics with predictive power that may then become the plan evaluation metrics for new plans.

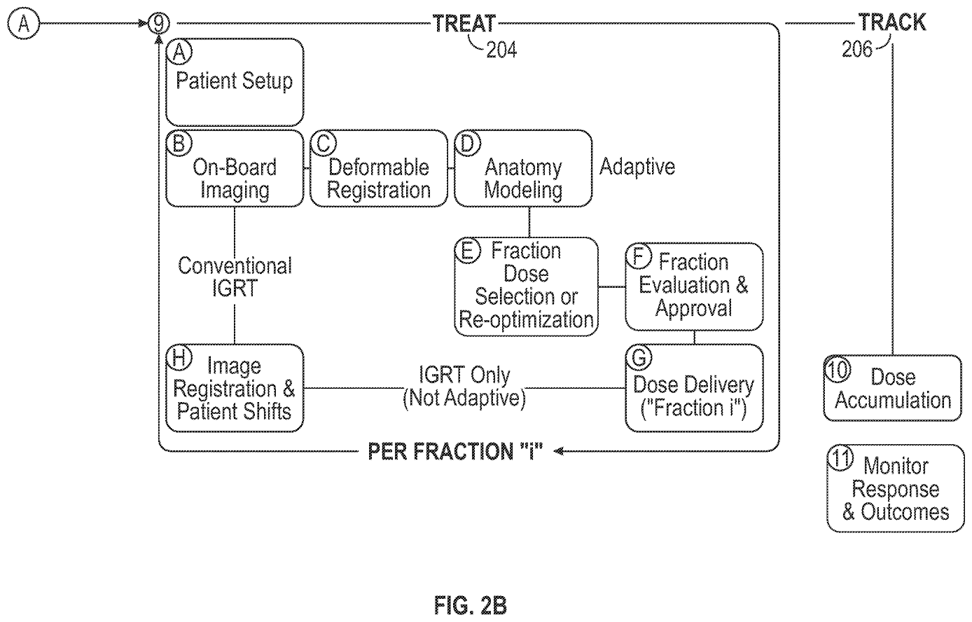

FIG. 3 is a graph illustrating an example of using patient-specific opportunities to personalize prescriptions for specific patient anatomies by sparing a healthy organic beyond generic protocol goals.

The graph 300 is an example of the first opportunity analysis use case described above with respect to FIGS. 2A and 2B. The graph 300 represents an example of an opportunity dose-volume histogram (ODVH) for an OAR, in accordance with this disclosure. The x-axis represents dose and the y-axis represents volume.

The dot 302 in the middle of the graph 300 represents a generic DVH-based wish list goal for an OAR. Essentially, any curve that is below that dot would be satisfactory. However, the present inventor has recognized that there may be opportunities to improve dose-sparing beyond the generic goal, and the degree of the opportunity is dependent on the patient's unique anatomy.

In FIG. 3, an “opportunity zone” is defined by two levels derived from a opportunity dose-volume histogram (ODVH) for one permutation of a patient's anatomy. One extraction (curve 304) represents a demarcation of what should be largely possible while another extraction (curve 306) captures the physical limit of what is possible for any specific beam modality (photon, proton, electron, or even internal brachytherapy sources).

The “opportunity zone” for this specific anatomy shows that this patient's OAR could be spared more (e.g., lower volume at dose, or lower dose at volume) than the generic goal asks for. In many OARs, the lower dose the better, even beyond the goal. A plan for this patient could be optimized for more sparing based on the unique patient anatomy and not just the generic protocol, i.e., optimized to a “personalized prescription.”

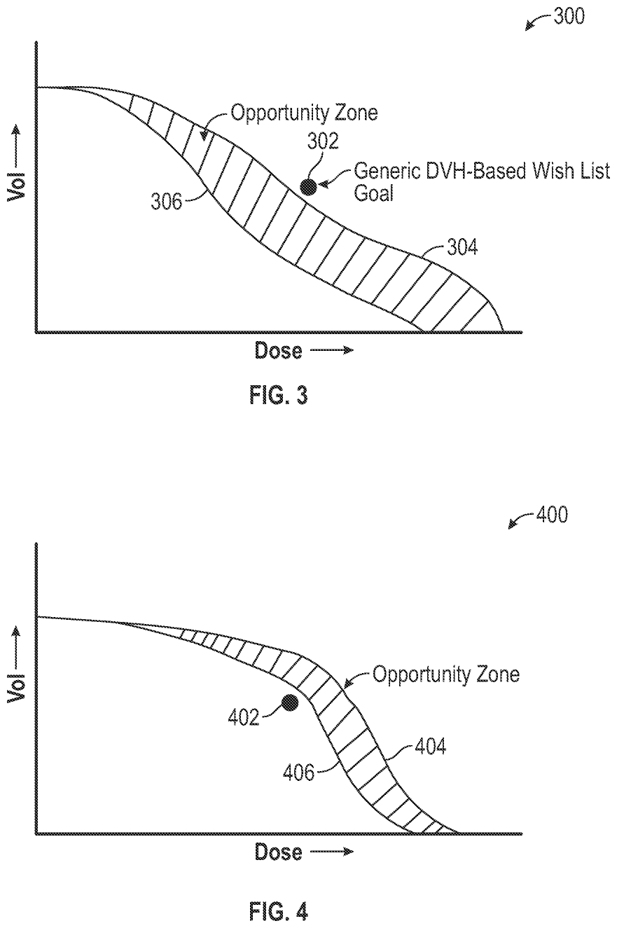

FIG. 4 is a graph illustrating another example of using patient-specific opportunities to personalize prescriptions, this time quantifying in advance any generic protocol goals that may be physically impossible. The graph 400 is another example of the first opportunity analysis use case described above with respect to FIGS. 2A and 2B. The graph 400 represents an example of an opportunity dose-volume histogram (ODVH) for an OAR, in accordance with this disclosure. The x-axis represents dose and the y-axis represents volume. Here, the outer opportunity limit (curve 404) and lower opportunity limit (curve 406) define an Opportunity Zone that is wholly above the generic protocol goal 402, indicating that conventional OAR sparing is probably not possible for this patient. If a planner tries to achieve the goal without realizing that the goal is impossible, the clinician (and/or the inverse-planning optimization engine) might spend a lot of time to achieve the dose sparing, yet keep failing. Instead, the planner might forewarn the physician and negotiate more lenient goals and acceptance criteria, which is another form of a personalized prescription, i.e., to relax conventional standards for a specific patient.

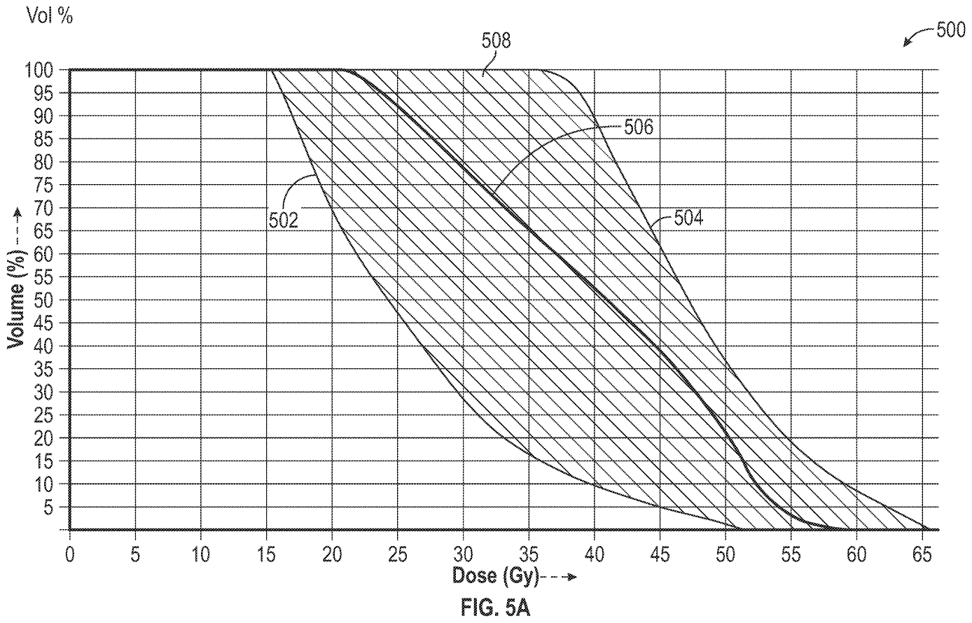

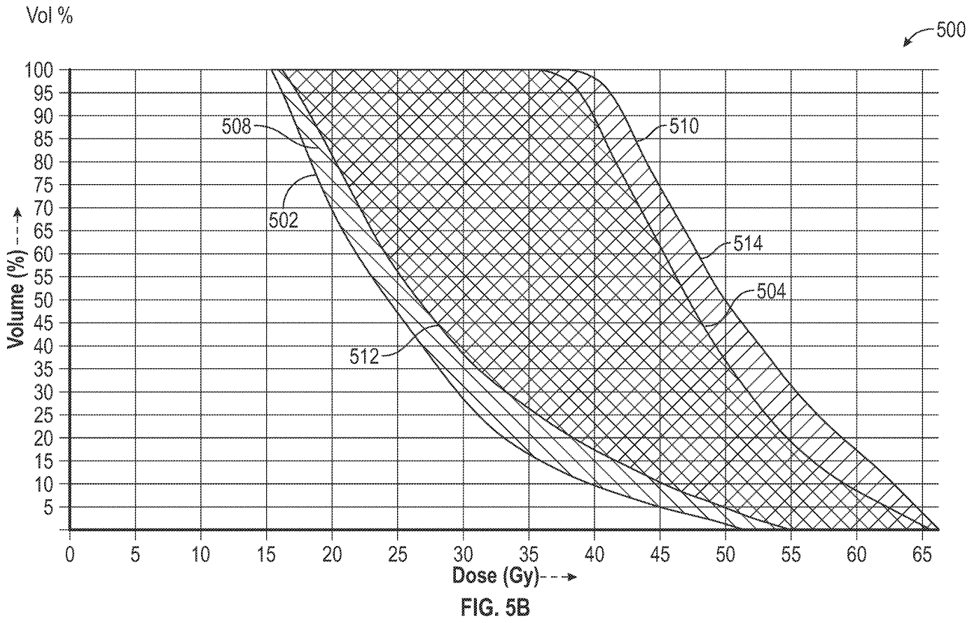

FIG. 5A is a graph depicting an example of an achieved/planned DVH compared to a patient-specific anatomy-based ODVH curve. The graph 500 is another example of the second opportunity analysis use case described above with respect to FIGS. 2A and 2B. The x-axis represents dose and the y-axis represents volume.

The line 502 represents a limit of sparing possible given the target's shapes and dose levels, or a lower opportunity zone limit. The second line 504 represents an upper limit. The region between the line 502 and the line 504 represents an opportunity zone 508, with the ability to move towards the line 502 giving higher resistance (more difficulty). Moving to the left of the line 502 is increasingly less physically realizable. As such, the opportunity zone 508 represents physically realizable dose distribution, in contrast to U.S. Pat. No. 9,463,336, which uses a fictitious treatment dose matrix that provides a quality of dose placement beyond that achievable with a physically realizable radiation therapy machine.

The line 506 represents the achieved/planned DVH. Plan evaluation allows a clinician to assess plan quality against the patient-specific OAR-sparing opportunities to supplement assessments based on generic protocol goals. Plan evaluation may generate and display ODVH curves against which to compare achieved DVH curves.

FIG. 5B is a graph depicting an example of the opportunities for sparing an OAR comparing one instance of a patient's anatomy to another instance (e.g., one treatment fraction versus another, or treatment fraction “i” versus the original plan designed based on the pre-treatment planning images). FIG. 5B depicts two overlapping ODVH zones for the same patient but for two different anatomical instances at different treatment fractions. The x-axis represents dose and the y-axis represents volume.

The graph of FIG. 5B includes the lower limit line 502 and upper limit line 504 of FIG. 5A overlapping with a second opportunity zone 510 in FIG. 5B. The second opportunity zone 510 is defined by a lower limit line 512 and an upper limit line 514, which in this example are different than the lower limit line 502 and upper limit line 504 of FIG. 5A. The second opportunity zone 510 has shifted (rightward) due to the changes in the patient's anatomy between the two instances, such as between different days. As seen by the rightward shift in the lower limit, there are fewer opportunities for sparing in this instance.

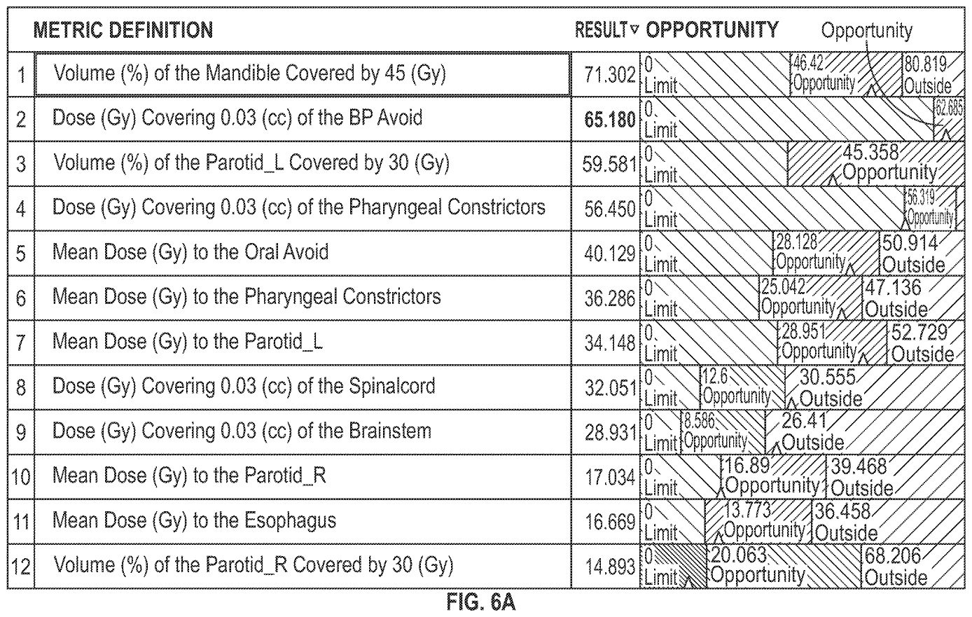

In addition, the plan evaluation techniques may generate personalized scorecards (“Opportunity Scorecards”) or tables as shown in FIG. 6A, that may be studied independently and also used to supplement generic protocol scorecards, as shown in FIG. 6B.

FIG. 6A depicts an example an opportunity scorecard, in accordance with this disclosure. The opportunity scorecard 600, e.g., a table, allows a clinician to analyze the achieved/planned results with an opportunity-based scorecard rather than generic goals. The opportunity scorecard 600 includes three columns. The lefthand column (“metric definition”) shows a label, such as a volume percentage of an OAR volume covered by a particular dose as in line 1. The middle column (“result”) shows the result, e.g., a numerical result of the dose received by a specific volume or volume irradiated by a specific dose. The righthand column (“opportunity”) graphically depicts a bar graph that includes the limit, the opportunity, and where the result of the middle column lies. For example, in line 5, although the achieved result for “mean dose to the ‘oral avoid’” OAR was 40.129 Gy, the opportunity for max-sparing is 28.128 Gy. As such, a clinician may graphically see the opportunity for further sparing of that specific OAR, if clinically warranted.

FIG. 6B shows examples of opportunity scorecard results viewed in conjunction with generic protocol goals. The scorecard 602, e.g., a table, shows how patient anatomy-specific opportunity scorecard results may be viewed in conjunction with generic protocol goals that might be used universally across all patients in a cohort.

The scorecard 602 includes four columns. The lefthand column 604 depicts the structure. Moving rightward, the column 604 depicts the metric to be extracted such as the volume percentage of the structure in column 602 covered by a particular dose, e.g., 45 Gy in FIG. 6B. The extracted metric could also be the dose covering a specific volume of the structure, or the minimum, mean, or maximum dose to that structure. The column 606 depicts two rows 610, that each correspond with the structure in column 602. The top row 610 depicts generic goals for the structure, given as performance bins, e.g., below 30 Gy being “ideal,” 30 to 35 Gy being “good,” all the way up to >50 Gy being “unacceptable.” The bottom row 612 depicts an opportunity band where the lower edge is the estimate if the OAR is max spared and the only priority, while the upper edge is an estimate of what should be achievable when all OARs are considered. Graphically speaking, these are the two intersections of the ODVH curve at any specific dose on the x-axis (if the metric is a dose value) or volume on the y-axis (if the metric is a volume). Column 608 depicts a label of which bin was achieved. For row 610, the result is a generic performance objective that would be extracted the same way for any patient. For row 612, it is a patient-specific performance on where the result occurred relative to its location inside (or potentially beyond) the opportunity band.

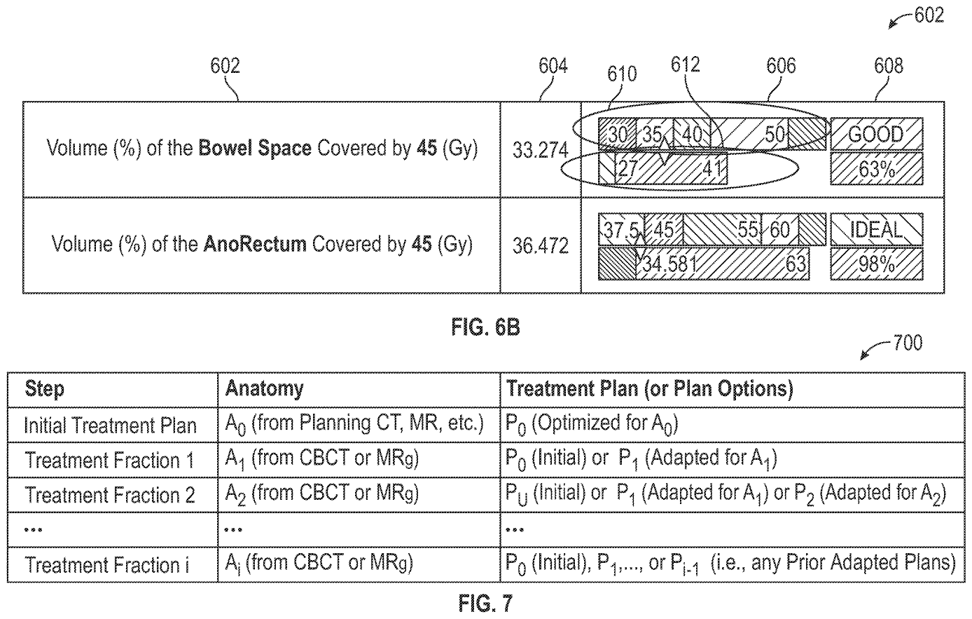

FIG. 7 is a chart 700 illustrating the relationship between steps, anatomy, and treatment plans (both initial and per-fraction/adaptive). The lefthand column represents the step. Steps include the initial treatment plan, treatment fraction 1, treatment fraction 2, and so forth until treatment fraction i.

The middle column represents a patient's anatomy. A 3D model of the patient's anatomy A₀ is designed for the initial treatment plan P₀, using CT, MR, etc. Then, a unique permutation of the patient's anatomy (anatomies A₁ through Aᵢ) is acquired before each treatment fraction using on-board imaging, e.g., cone-beam CT (CBCT) or MR-guidance (MRg).

The righthand column represents the treatment plan. The initial treatment plan P₀ is optimized for anatomy A₀. After each fraction's image, there is a basic decision for the clinician: (1) use the initial treatment plan P₀, or (2) use an adapted treatment plan from a prior fraction, or (3) optimize a new adapted plan. For example, at treatment fraction 1, the clinician may determine whether to use the initial treatment plan P₀, or use a new adapted plan P₁ that was adapted for the anatomy A₁ of treatment fraction 1. At treatment fraction 2, the clinician may determine whether to use the initial treatment plan P₀, or use the previously adapted plan P₁ that was adapted for the anatomy A₁ of treatment fraction 1, or use a new adapted plan P₂ that was adapted for the anatomy A₂ of treatment fraction 2. At treatment fraction i, the clinician determines whether to use the initial treatment plan P₀, or use any of the prior adapted plans P₁, P₂, or use a new adapted plan Pᵢ that was adapted for the anatomy Aᵢ of treatment fraction i.

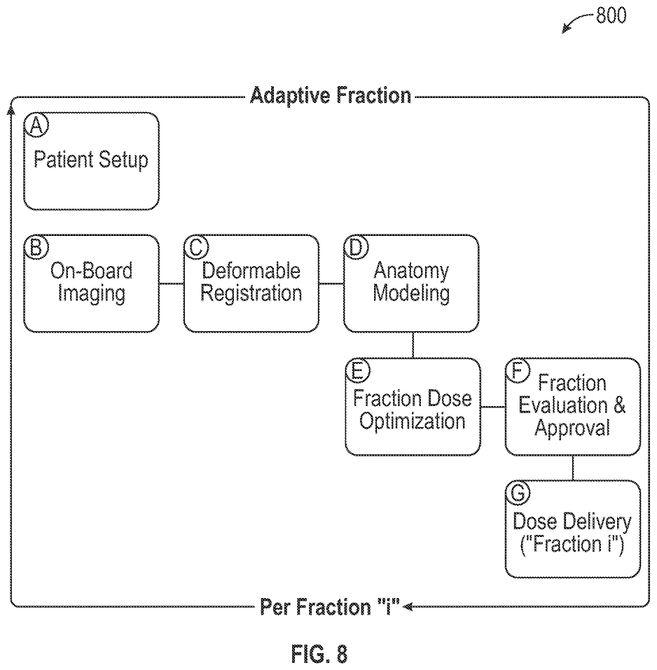

FIG. 8 is a flow diagram illustrating an adaptive radiotherapy planning process 800 utilizing the adaptive opportunity assistance techniques of this disclosure. The adaptive opportunity assistance techniques were mentioned above as the third opportunity analysis use case with respect to FIGS. 2A and 2B. FIG. 8 depicts seven steps in the process 800 including patient setup (step A), on board imaging (step B), deformable registration (step C), anatomy modeling (step D), fraction dose optimization (step E), fraction evaluation and approval (step F), dose delivery (“Fraction i”)(step G). The adaptive opportunity assistance techniques may occur in the fraction dose optimization block (step E).

Using existing approaches to adaptive radiation therapy, a clinician optimizes a new adapted plan, evaluates the adapted plan, and then iterates until satisfied with the adapted plan. These existing approaches present at least two problems. First, it is time-consuming, especially considering that for “online” adaptive therapy, the patient is immobilized on a treatment table. Second, the newly adapted plan uses optimization criteria of the initial plan based on the anatomy A₀, and may not take advantage of new opportunities for OAR dose-sparing based on unique anatomy Aᵢ (or similarly, may not know the dose-sparing achieved prior is not possible for the new anatomy instance).

The adaptive opportunity assistance techniques of this disclosure provide an improved process. The adaptive opportunity assistant establishes a current fraction's new opportunity based on the unique anatomy of the day. The adaptive opportunity assistant may compare today's anatomy across all opportunity dose grids from the initial plan and for each of any prior fractions adapted plan. Then, if there is a sufficient match based on advanced opportunity cost metrics, that plan may be a candidate to use for this fraction. However, if the match is not sufficient, the clinician now has confidence that they need to optimize a new adaptive plan.

The adaptive opportunity assistance techniques of this disclosure provide several benefits. First, the process will save time in the aggregate (i.e., over all treatment fractions) versus always adapting a new plan by default. Second, if the clinician does decide to optimize an adaptive plan, they may use the personalized prescription techniques to optimize sparing for that fraction instead of an optimization template based on a prior (and different) anatomy of the patient.

The adaptive opportunity assistant may provide a quantified and rapid decision support for adaptive radiation therapy. The adaptive opportunity assistant may provide rapid assessment of the sparing opportunity for fraction i versus the initial plan and versus prior fraction(s). In addition, the adaptive opportunity assistant may provide rapid analysis of the initial plan and over the library of prior fractions' adaptive plans to look for the best matches. Further, if a new adaptive plan is warranted, the adaptive opportunity assistant may help in understanding this fraction's unique opportunities (e.g., a personalized prescription for that specific fraction).

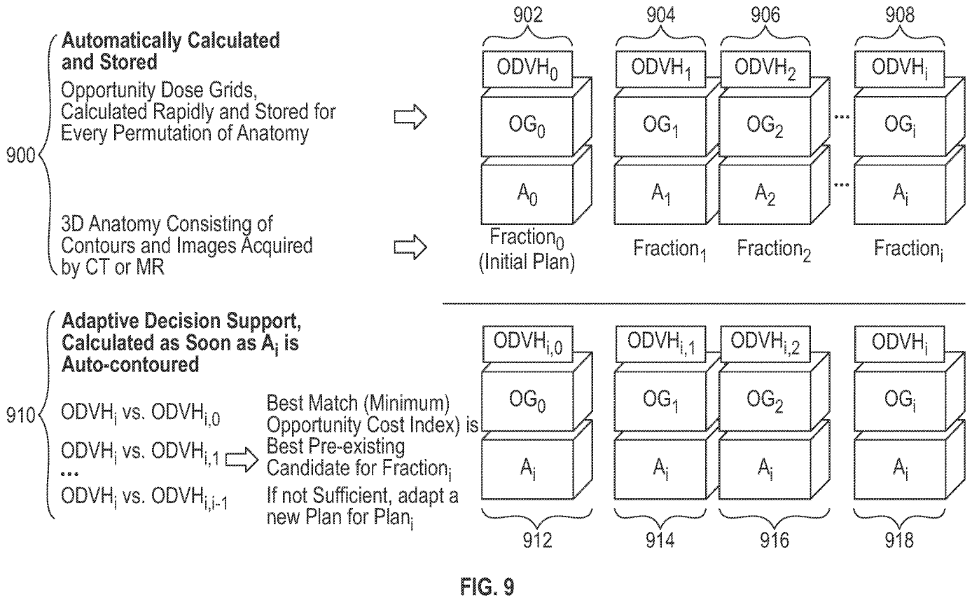

FIG. 9 is a conceptual diagram illustrating an example of an adaptive opportunity assistant for a treatment fraction “i”. The top half 900 of FIG. 9 illustrates that the adaptive opportunity assistant may generate the maximum dose-sparing opportunity for a particular treatment session. The anatomy-specific opportunity grids and opportunity dose-volume histograms (ODVH) are automatically calculated and stored. The column 902 of the top half 900 represents Fraction₀ (the initial plan) based on anatomy A₀ (the initial planning anatomy). The column 904 of the top half 900 represents Fraction₁ (for anatomy A₁), the column 906 of the top half 900 represents Fraction₂ (for anatomy A₂), and the column 908 of the top half 900 represents Fractionᵢ (for anatomy Aᵢ). The opportunity dose grids are calculated rapidly and stored for every permutation of anatomy A₀-Aᵢ for a corresponding Fraction₀-Fractionᵢ. The 3D anatomy A includes contours and images acquired by CT or MR. A corresponding ODVH is determined for each Fraction, e.g., ODVH₀ for Fraction₀, ODVH₁ for Fraction₁, and so forth. Each day's anatomy is paired with that day's opportunity dose grid (OG).

A representation of the maximum dose-sparing opportunity for a particular treatment session may be displayed to the user, e.g., clinician. In response to receiving an input representing a selection, the radiotherapy system 100 may modify, based on the displayed output, the radiotherapy treatment plan for the current radiotherapy treatment fraction.

The bottom half 910 of FIG. 9 illustrates that the adaptive opportunity assistant may provide adaptive decision support by examining any/all of the anatomy-based opportunity grids for any corresponding previous imaging and/or treatment sessions. During a fraction, the adaptive opportunity assistant may analyze any previously adapted plans from prior fractions to determine if the anatomy-specific opportunity is “close enough” to the opportunity calculated for the current treatment session, where “close enough” would be determined by a tolerance in the Opportunity Cost metric(s). If a match is found, that previously generated treatment plan (already optimized for the prior fraction's anatomy and opportunity) may be retrieved and re-used, which may save considerable time when compared with optimizing a plan. If not, the clinician may decide whether the dose-sparing opportunity is significant enough to warrant adapting or re-optimizing to create a new treatment plan for the current fraction.

The column 912 of the bottom half 910 represents the dose-sparing opportunity provided by current Fractionᵢ compared against the target coverage and dose-sparing opportunity provided by the initial treatment plan of Fraction₀, designated by ODVH_{i,0}.

The column 914 of the bottom half 910 represents the target coverage and dose-sparing opportunity provided by the current Fractionᵢ compared against the dose-sparing opportunity provided by the treatment plan of the first Fraction₁, designated by ODVH_{i,1}.

The column 916 of the bottom half 910 represents the target coverage and dose-sparing opportunity provided by current Fractionᵢ compared against the dose-sparing opportunity provided by the treatment plan of the second Fraction₂, designated by ODVH_{i,2}.

The column 918 of the bottom half 910 is the same as column 908 of the top half 900 of FIG. 9 and represents the target coverage and maximum dose-sparing opportunity for the current Fractionᵢ designated by ODVHᵢ. This current ODVHᵢ is compared against the ODVHs of previously generated plans to generate ODVH_{i,0}, ODVH_{i,1}, and ODVH_{i,2} of columns 912-914. A best match, e.g., minimum opportunity cost index, is the best pre-existing candidate for Fractionᵢ. Various opportunity cost metrics are described below.

The radiotherapy system 100 may receive a selection, based on a displayed output, such as shown in FIG. 9, between an original radiotherapy treatment plan and a radiotherapy treatment plan generated subsequent to the original radiotherapy treatment plan. In some examples, the radiotherapy treatment plan generated subsequent to the original radiotherapy treatment plan was developed during a radiotherapy treatment fraction prior to a current radiotherapy treatment fraction.

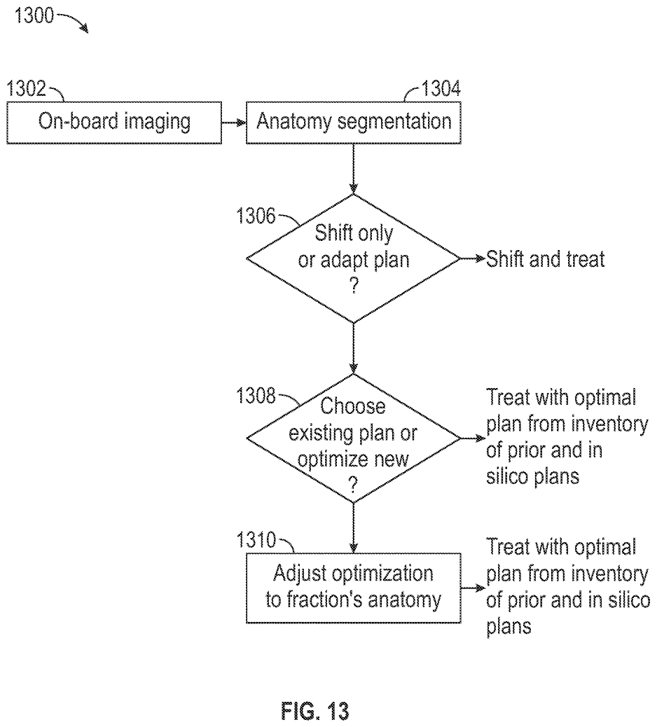

FIG. 13 is a flow diagram illustrating an example of an adaptive radiotherapy planning process 1300 utilizing opportunity-assisted decision support techniques in accordance with this disclosure. The adaptive radiotherapy planning process 1300 may begin with on-board imaging 1302 followed by anatomy segmentation 1304.

At block 1306, the adaptive radiotherapy planning process 1300 determines whether to “shift only” or adapt the plan. For example, using Opportunity Assistance, the radiotherapy system may analyze new anatomy with respect to the initial anatomy using the Opportunity Grid (described above) for the initial plan to see if that initial plan is well-suited for target coverage and OAR sparing. If the system determines that they are sufficiently matched, then “shift only” is a valid option. For example, the system may compare ODVH (i) versus ODVH (i,0) and use Opportunity Cost metrics to determine how closely they match, such as in a priority-weighted aggregate. If they are not sufficiently close, then the system may choose an existing plan or optimize a new plan at block 1308.

At block 1308, the adaptive radiotherapy planning process 1300 determines whether to choose an existing plan or optimize a new plan. Using Opportunity Assistance, the system may analyze the inventory of anatomy permutations (from prior fractions and/or from simulations) and their pre-designed plans to see if there is an already-designed plan that is well-matched to the current fraction's anatomy. For example, the system may compare ODVH (i) versus ODVH (i,j) where j is each item in the inventory. The system may use Opportunity Cost metrics to determine how closely they match, such as in a priority-weighted aggregate. If they are not sufficiently close, then the system may select an existing plan. If not, the system may optimize a new plan at block 1310.

At block 1310, the adaptive radiotherapy planning process 1300 adjusts optimization parameters (inputs to the inverse-planning engine) to the fraction's unique anatomy. Using Opportunity Assistance, the system may establish a current fraction's ODVHs based on the day's anatomy and compare to the reference plan's ODVHs to know how to adjust each OAR-specific objective. For example, the system may compare ODVH (i) versus ODVH (0) and use Opportunity Cost metrics (e.g., average dose or volume shifts) to adjust the initial optimization parameters according to the new permutation of anatomy.

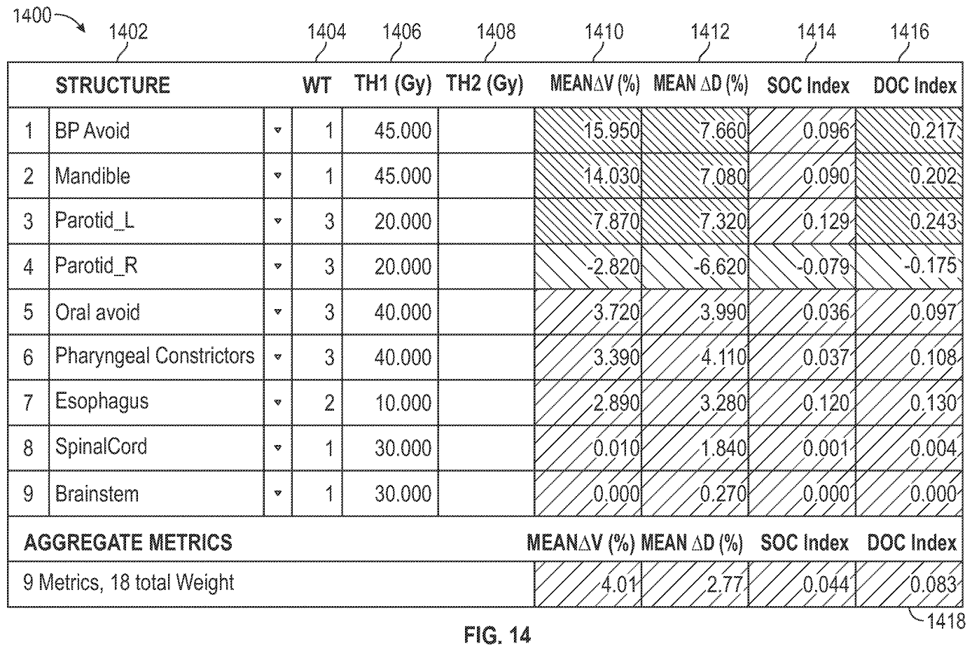

FIG. 14 depicts an example of a scorecard depicting an aggregation of opportunity costs over multiple OARs for plan evaluation, in accordance with this disclosure. The scorecard 1400, e.g., a table, allows a clinician to analyze simple opportunity cost metrics (or “SOC”) and the dose-weighted opportunity cost metrics (or “DOC”) results for each OAR as well as an aggregation of opportunity costs over multiple OARs.

The opportunity scorecard 1400 shown includes eight columns. The lefthand column 1402 (“structure”) shows an OAR. Moving to the right, the next column 1404 depicts a weight (or priority weight) that may be assigned to the structure, e.g., from 1-3. The column 1406 depicts a lower threshold for consideration of opportunity costs, in units of radiation dose (Gy). The column 1408 depicts an upper dose threshold, which can be optional and assumed unbounded. The column 1410 depicts a mean ΔV %, or mean change in volume. The column 1412 depicts a mean ΔD %, or mean change in radiation dose. The column 1414 depicts the SOC index for the OAR. The righthand column 1414 depicts the DOC index for the OAR.

Aggregate quality metrics for the columns 1410-1416 are shown at row 1418. Aggregate quality metrics may be calculated across all critical OARs, with per-OAR priority weighting, e.g., weighted average of all SOC indices, DOC indices, etc. Examples include the following:
(1) Achieved DVH for intended plan (comparison) versus ODVH band (reference);
(2) Achieved DVH for intended plan (comparison) versus 3rd party predicted DVH or feasibility DVH (reference);
(3) ODVH for fraction “i” (comparison) versus ODVH for the intended plan fraction (reference);
(4) ODVH for fraction “i” (comparison) versus ODVH for a prior plan fraction (reference); and
(5) Achieved DVH for modality B (comparison) versus Achieved DVH for modality A (reference).

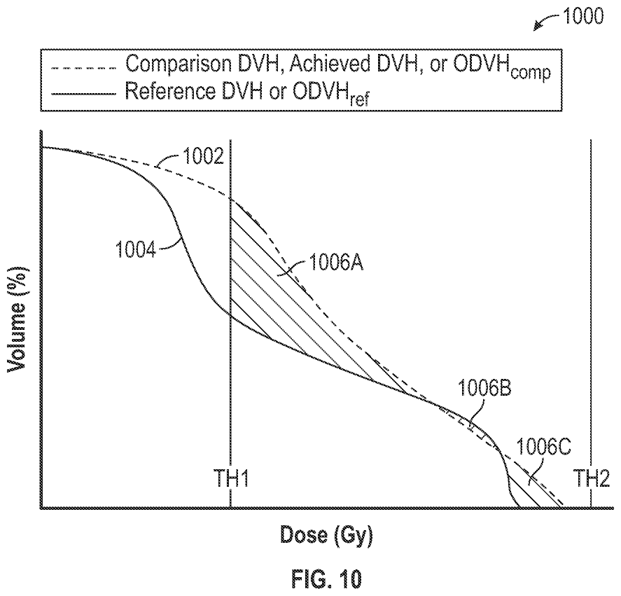

FIG. 10 depicts a graph illustrating an example of evaluating a radiotherapy dose-sparing opportunity for a subject. FIG. 10 illustrates a difference between a first ODVH and a second ODVH, where the difference represents a radiotherapy dose-sparing opportunity for a specific organ at risk (OAR). The graph 1000 illustrates an extracted opportunity level from the first ODVH 1002, e.g., a reference DVH (ODVH_{ref}), such as modelled based on an anatomy of the patient in a first imaging or radiotherapy treatment session and a second ODVH 1004, e.g., a comparison DVH (ODVH_{comp}), such as modelled based on an anatomy of the patient in a second imaging or radiotherapy treatment session. In this disclosure, the terms “first” and “second” do not imply a temporal relationship; the first imaging or radiotherapy treatment session may occur before or after the second imaging or radiotherapy treatment session. The x-axis represents dose and the y-axis represents volume.

In the example of FIG. 10, two dose thresholds, the minimum dose threshold TH1 and a maximum dose threshold TH2, are shown. In some examples, the two dose thresholds may be inputs, such as to the radiotherapy system 100 of FIG. 1, that define a dose range of interest (e.g., a clinically-relevant dose range that is a subset of the entire dose range from 0 Gy to maximum dose). In other examples, the radiotherapy system 100 may receive just one threshold, such as a minimum dose of interest, e.g., the minimum threshold TH1, and the upper threshold is not defined, and thus considered unbounded, always including up to the maximum dose observed for that OAR in either the reference or comparison curve.

The difference between the first ODVH 1002 and the second ODVH 1004 is represented by regions 1006A-1006C, where the difference represents the dose-sparing opportunity for the subject, e.g., patient. In some examples, the difference between the first ODVH 1002 and the second ODVH 1004 is a difference between a representation of an area under a curve of the first ODVH 1002 and a representation of an area under a curve of the second ODVH 1004.

The radiotherapy system 100 may generate an output that represents the determined difference, and then display the output to a user, such as via the display device 134. If the clinician determines that the difference, e.g., the sum of the regions 1006A-1000C, is sufficient, then the time-consuming process of modifying the treatment plan may be warranted because of the dose-sparing. In some examples, the displayed output may be similar to the graph shown in FIG. 10. In other examples, the displayed output may be a number representing the “opportunity cost”, such as using one of the equations described below.

In some examples, the radiotherapy system 100 may weight the determined difference using the corresponding radiotherapy dose level on the x-axis. For example, the differences represented by the region 1006C may be weighted more heavily than the differences represented by the region 1006A given that a higher dose is applied to the volume in the region 1006C. In some examples, the radiotherapy system 100 may normalize the weighted difference.

In some examples, the output displayed represents a volume-specific output, e.g., a bowel space. In other examples, the output displayed represents an organ-specific output, e.g., the anorectum. In other examples, the output displayed represents an aggregation of multiple OARs, rather than a single OAR. In some examples, the radiotherapy system 100 may weight the output based on an importance of the OAR. For example, a clinician may rank various OARs based on their importance and the radiotherapy system 100 may weight the output based on their ranking. Weighted outputs may also be summed to produce an aggregate value that encompasses all relevant OARs, weighted by user-defined importance.

The radiotherapy system 100, or some other machine, may determine the difference between the first ODVH 1002 and the second ODVH 1004 using various unweighted or dose-weighted “opportunity cost” metrics, such as but not limited to those represented by Equations 1-5 below. A three-dimensional (3D) dose and volume may be reduced to a two-dimensional (2D) figure using DVH curves, such as shown in FIG. 10. Using the opportunity cost metrics, two 2D DVH curves may be reduced to a number, such as for display to a clinician. The clinician may easily interpret the number.

In a first example, the difference between the first ODVH 1002 and the second ODVH 1004 may be determined using a simple opportunity cost metric (or “SOC”), as shown in Equation 1:

$$SOC = {\int }_{TH 1}^{TH 2} [ {V}_{Comp} ( D ) - {V}_{Ref} ( D ) ] \cdot dD \qquad Equation 1$$

The SOC metric is the difference in area under the two curves between the dose thresholds TH1, TH2, with units of dose times volume (Gy·% or Gy·cm³).

In a second example, the difference between the first ODVH 1002 and the second ODVH 1004 may be determined using Equation 2:

$$\overline{Δ Vol} = \frac{SOC}{[ D 2 - D 1 ]} \qquad Equation 2$$

Equation 2 is the mean volume difference and may be calculated using the SOC from Equation 1 divided by the desired dose range (TH2−TH1).

In a third example, the difference between the first ODVH 1002 and the second ODVH 1004 may be determined using Equation 3:

$$SOC Index = SOC / {\int }_{0}^{Max} {V}_{Ref} ( D ) \cdot dD \qquad Equation 3$$

Equation 3 is the SOC index, which is a normalized metric determined by dividing the SOC from Equation 1 by the area of the reference curve over the whole dose range.

In a fourth example, the difference between the first ODVH 1002 and the second ODVH 1004 may be determined using Equation 4:

$$DOC = {\int }_{D 1}^{D 2} D \cdot [ {V}_{Comp} ( D ) - {V}_{Ref} ( D ) ] \cdot dD \qquad Equation 4$$

A higher dose is more damaging than a lower dose and Equation 4 weights the volumes accordingly. Equation 4 is the dose-weighted opportunity cost metric (or “DOC”), with units of dose squared times volume (Gy²·% or Gy²·cm³).

In a fifth example, the difference between the first ODVH 1002 and the second ODVH 1004 may be determined using Equation 5:

$$DOC Index = DOC / {\int }_{0}^{Max} D \cdot {V}_{Ref} ( D ) \cdot dD \qquad Equation 5$$

The DOC index of Equation 5 is a normalized metric determined by dividing the DOC from Equation 4 by the area of the dose-weighted reference curve over the whole dose range.

Using these techniques of this disclosure, two ODVHs may be determined from two different imaging or radiotherapy treatment session. Then, a radiotherapy dose-sparing opportunity for an OAR may be determined based on a difference between the two ODVHs. An output that represents the radiotherapy dose-sparing opportunity for the OAR may then be generated and displayed to a user, e.g., clinician. Based on the displayed output, the user may be able to quickly determine the new clinically significant sparing opportunities that are available, and adjust the optimization parameters for the adaptive plan rather than using prior parameters. Or, if the ODVHs are very similar, the prior parameters can be used again when directing the optimizer. To adapt a new plan, it is implied that it is worth the time and effort, and the potential additional discomfort to the subject, e.g., patient, to create a new plan in real-time (if online adaptive) or to use more staff time to generate a new treatment plan based on anatomy changes between fractions (if offline adaptive). However, it is also of great use to know if you need to adapt a new plan at all, or if the initial (or any prior) plan is sufficient. In this case, the opportunity grids of the initial and prior plans can be matched to the new fraction's anatomy, and if a good match is found (i.e., opportunity costs within a tolerance specified by the clinicians), to use that existing plan rather than optimize a new one.

In addition to the benefits described above, the techniques of this disclosure allow dose-sparing to be accumulated over the course of radiotherapy treatment. Some treatment fractions may not have any clinically significant dose-sparing opportunities and, in some fractions, the OAR may receive a higher dose than the generic goal due to the unique anatomy of that day. But, with the techniques of this disclosure, dose-sparing may accumulate over the course of treatment and partially offset or, in some instances, more than offset the fractions where the OAR received a higher dose than the generic goal. In this manner, the OAR may receive a lower total dose than the generic goal over the course of treatment, despite the OAR receiving a higher dose than the generic goal during a particular fraction.

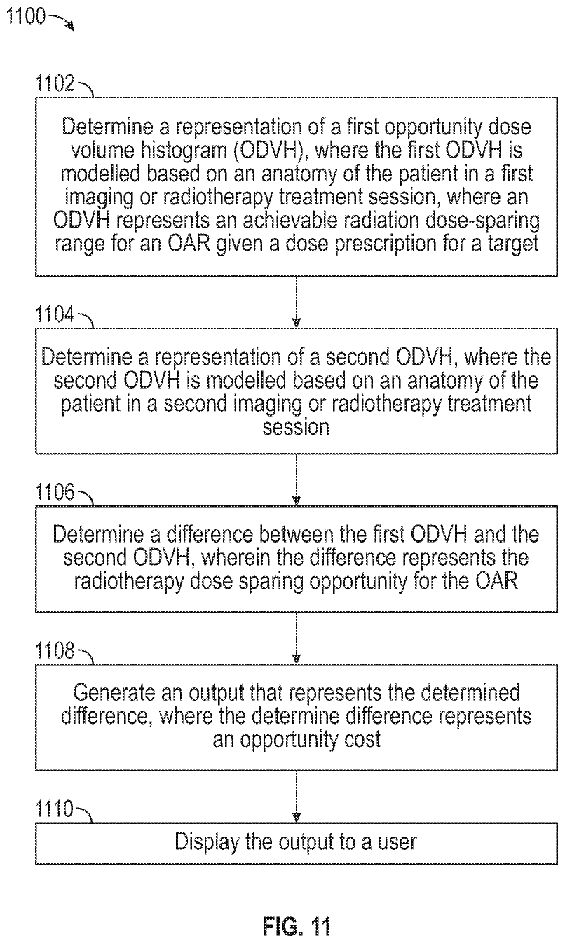

FIG. 11 is a flow diagram of an example of a computer-implemented method 1100 of analyzing a radiotherapy dose-sparing opportunity for a subject. At block 1102, the method 1100 may include determining, using computer processor circuitry, a representation of a first opportunity dose-volume histogram (ODVH), where the first ODVH is modelled based on an anatomy of the patient in a first imaging or radiotherapy treatment session, where an ODVH represents an achievable radiation dose-sparing range for an organ at risk (OAR) given a dose prescription for a target.

At block 1104, the method 1100 may include determining, using the computer processor circuitry, a representation of a second ODVH, where the second ODVH is modelled based on an anatomy of the patient in a second imaging or radiotherapy treatment session.

At block 1106, the method 1100 may include determining, using the computer processor circuitry, a difference between the first ODVH and the second ODVH, where the difference represents the radiotherapy dose-sparing opportunity for the OAR.

At block 1108, the method 1100 may include generating, using the computer processor circuitry, an output that represents the determined difference, where the determined difference represents an opportunity cost.

At block 1110, the method 1100 may include displaying the output to a user.

In some examples, the output represents a volume-specific output. In other examples, the output represents a dose-specific opportunity cost.

In some examples, the output represents an aggregation of opportunity costs over multiple OARs.

In some examples, the method 1100 includes weighting the output based on an importance of the OAR.

In some examples, the method 1100 includes receiving an input representing a minimum dose of interest, such as on its own or as part of a range. In some examples, the method 1100 includes receiving an input that defines a dose range of interest.

In some examples, determining the difference between the first ODVH and the second ODVH includes determining a difference between a representation of an area under a curve of the first ODVH and a representation of an area under a curve of the second ODVH. In some examples, the method 1100 includes weighting the determined difference using a corresponding radiotherapy dose. In some examples, the method 1100 includes normalizing the weighted determined difference.

In some examples, the first ODVH corresponds to a previous radiotherapy treatment fraction, and the second ODVH corresponds to a current radiotherapy treatment fraction, and the method 1100 includes modifying, based on the displayed output, a radiotherapy treatment plan for the current radiotherapy treatment fraction.

In some examples, the first ODVH corresponds a prior imaging and/or treatment session, and wherein the second ODVH corresponds to a current radiotherapy treatment fraction, and the method 1100 includes modifying, based on the displayed output, the radiotherapy treatment plan for the current radiotherapy treatment fraction.

In some examples, the method 1100 includes receiving a selection, based on the displayed output, between an original radiotherapy treatment plan and a radiotherapy treatment plan generated subsequent to the original radiotherapy treatment plan. In some examples, the radiotherapy treatment plan generated subsequent to the original radiotherapy treatment plan was developed during a radiotherapy treatment fraction prior to a current radiotherapy treatment fraction.

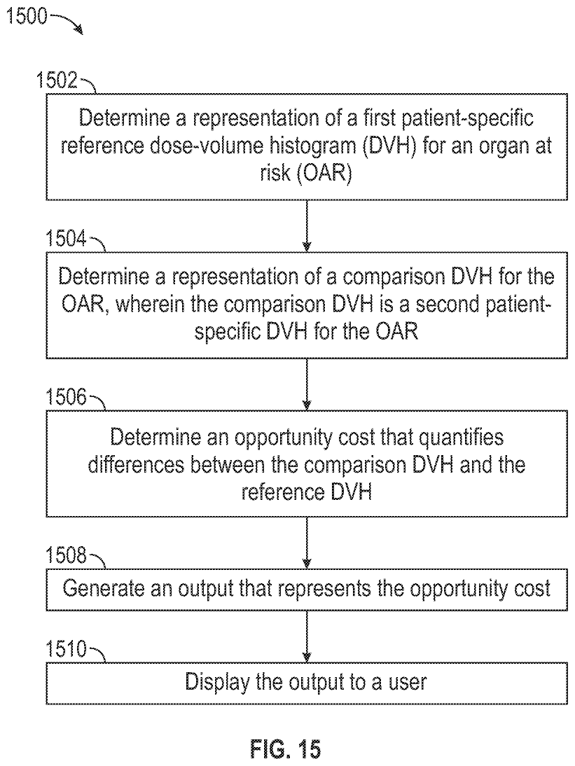

The techniques described in this disclosure, including those in FIGS. 11 and 15, may be implemented using the system radiotherapy system 100 of FIG. 1 or the machine 1200 of FIG. 12, for example.

FIG. 15 is a flow diagram of an example of a computer-implemented method 1500 of evaluating a radiotherapy plan for a patient. At block 1502, the method 1500 may include determining, using computer processor circuitry, a representation of a first patient-specific reference dose-volume histogram (DVH) for an organ at risk (OAR). The reference DVH is an input DVH against which another DVH will be compared. In some examples, the reference DVH is the lower band of an ODVH, but it could also be a DVH from a competing modality (e.g., proton plan against which to judge a photon plan), or an achieved or predicted DVH imported from an outside system. At block 1504, the method 1500 may include determining, using the computer processor circuitry, a representation of a comparison DVH for the OAR, wherein the comparison DVH is a second patient-specific DVH for the OAR. In some examples, the second DVH is a planned DVH that is being judged for quality compared to the reference DVH.

At block 1504, the method 1500 may include determining, using the computer processor circuitry, an opportunity cost that quantifies differences between the comparison DVH and the reference DVH.

At block 1504, the method 1500 may include generating, using the computer processor circuitry, an output that represents the opportunity cost.

At block 1504, the method 1500 may include displaying the output to a user.

In some examples, the opportunity cost is determined by an opportunity cost metric. In some examples, the method 1500 may include normalizing, using the computer processor circuitry, the opportunity cost metric.

In some examples, determining the opportunity cost includes determining a mean volume difference. In some examples, determining the opportunity cost includes determining a dose-weighted opportunity cost.

In some examples, the reference DVH and the comparison DVH are defined across a continuous dose range defined by a lower dose threshold and not by discrete points.

In some examples, the output represents an aggregation of opportunity costs over multiple OARs, such as priority weighted OARs.

FIG. 12 illustrates a block diagram of an embodiment of a machine 1200 on which one or more of the methods as discussed herein may be implemented. In one or more embodiments, one or more items of the image processing device 112 may be implemented by the machine 1200. In alternative embodiments, the machine 1200 operates as a standalone device or may be connected (e.g., networked) to other machines. In one or more embodiments, the image processing device 112 may include one or more of the items of the machine 1200. In a networked deployment, the machine 1200 may operate in the capacity of a server or a client machine in server-client network environment, or as a peer machine in a peer-to-peer (or distributed) network environment. The machine 1200 may be a personal computer (PC), a tablet PC, a set-top box (STB), a Personal Digital Assistant (PDA), a cellular telephone, a web appliance, a network router, switch or bridge, or any machine capable of executing instructions (sequential or otherwise) that specify actions to be taken by that machine. Further, while only a single machine 1200 is illustrated, the term “machine” shall also be taken to include any collection of machines that individually or jointly execute a set (or multiple sets) of instructions to perform any one or more of the methodologies discussed herein.

The example machine 1200 includes processing circuitry (e.g., the processor 1202, a CPU, a GPU, an ASIC, circuitry, such as one or more transistors, resistors, capacitors, inductors, diodes, logic gates, multiplexers, buffers, modulators, demodulators, radios (e.g., transmit or receive radios or transceivers), sensors 1221 (e.g., a transducer that converts one form of energy (e.g., light, heat, electrical, mechanical, or other energy) to another form of energy), or the like, or a combination thereof), a main memory 1204 and a static memory 1206, which communicate with each other via a bus 1208. The machine 1200 (e.g., computer system) may further include a video display unit 1210 (e.g., a liquid crystal display (LCD) or a cathode ray tube (CRT)). The machine 1200 also includes an alphanumeric input device 1212 (e.g., a keyboard), a user interface (UI) navigation device 1214 (e.g., a mouse), a disk drive or mass storage unit 1216, a signal generation device 1218 (e.g., a speaker), and a network interface device 1220.

The disk drive or mass storage unit 1216 includes a machine-readable medium 1222 on which is stored one or more sets of data structures and instructions (e.g., software) 2024 embodying or utilized by any one or more of the methodologies or functions described herein. The instructions 1224 may also reside, completely or at least partially, within the main memory 1204 and/or within the processor 1202 during execution thereof by the machine 1200, the main memory 1204 and the processor 1202 also constituting machine-readable media.

The machine 1200 as illustrated includes an output controller 1226. The output controller 1226 manages data flow to/from the machine 1200. The output controller 1226 is sometimes called a device controller, with software that directly interacts with the output controller 1226 being called a device driver.

While the machine-readable medium 1222 is shown in an embodiment to be a single medium, the term “machine-readable medium” may include a single medium or multiple media (e.g., a centralized or distributed database, and/or associated caches and servers) that store the one or more instructions 1224 or data structures. The term “machine-readable medium” shall also be taken to include any tangible medium that is capable of storing, encoding, or carrying instructions for execution by the machine and that cause the machine to perform any one or more of the methodologies of the present disclosure, or that is capable of storing, encoding, or carrying data structures utilized by or associated with such instructions. The term “machine-readable medium” shall accordingly be taken to include, but not be limited to, solid-state memories, and optical and magnetic media. Specific examples of machine-readable media include non-volatile memory, including by way of example semiconductor memory devices, e.g., Erasable Programmable Read-Only Memory (EPROM), EEPROM, and flash memory devices; magnetic disks such as internal hard disks and removable disks; magneto-optical disks; and CD-ROM and DVD-ROM disks.

The instructions 1224 may further be transmitted or received over a communications network 1228 using a transmission medium. The instructions 1224 may be transmitted using the network interface device 1220 and any one of a number of well-known transfer protocols (e.g., HTTP). Examples of communication networks include a LAN, a WAN, the Internet, mobile telephone networks, Plain Old Telephone (POTS) networks, and wireless data networks (e.g., WiFi and WiMax networks). The term “transmission medium” shall be taken to include any intangible medium that is capable of storing, encoding, or carrying instructions for execution by the machine, and includes digital or analog communications signals or other intangible media to facilitate communication of such software.

As used herein, “communicatively coupled between” means that the entities on either of the coupling must communicate through an item therebetween and that those entities cannot communicate with each other without communicating through the item.

### Various Aspects

Various aspects of this disclosure are directed to, among other things, opportunity analyses for adaptive planning for radiotherapy systems and opportunity analyses for adaptive planning for radiotherapy systems and opportunity analyses for plan evaluation for radiotherapy systems. Opportunity analyses for adaptive planning for radiotherapy systems may include the following number Aspects:

Aspect 1 can include or use subject matter (e.g., a system, apparatus, method, article, or the like) that can include or use a computer-implemented method of analyzing a radiotherapy dose-sparing opportunity for a subject, the computer-implemented method comprising: determining, using computer processor circuitry, a representation of a first opportunity dose-volume histogram (ODVH), wherein the first ODVH is modelled based on an anatomy of the patient in a first imaging or radiotherapy treatment session, and wherein an ODVH represents an achievable radiation dose-sparing range for an organ at risk (OAR) given a dose prescription for a target; determining, using the computer processor circuitry, a representation of a second ODVH, wherein the second ODVH is modelled based on an anatomy of the patient in a second imaging or radiotherapy treatment session; determining, using the computer processor circuitry, a difference between the first ODVH and the second ODVH, wherein the difference represents the radiotherapy dose-sparing opportunity for the OAR; generating, using the computer processor circuitry, an output that represents the determined difference, wherein the determined difference represents an opportunity cost; and displaying the output to a user.

Aspect 2 can include or use or can optionally be combined with at least some features of Aspect 1 to include or use the computer-implemented method wherein the output represents a volume-specific opportunity cost.

Aspect 3 can include or use or can optionally be combined with at least some features of Aspect 1 to include or use the computer-implemented method wherein the output represents a dose-specific opportunity cost.

Aspect 4 can include or use or can optionally be combined with at least some features of Aspect 1 to include or use the computer-implemented method wherein the output represents an aggregation of opportunity costs over multiple OARs.

Aspect 5 can include or use or can optionally be combined with at least some features of Aspect 1 to include or use the computer-implemented method further comprising: weighting the output based on an importance of the OAR.

Aspect 6 can include or use or can optionally be combined with at least some features of Aspect 1 to include or use the computer-implemented method further comprising: receiving an input representing a minimum dose of interest.

Aspect 7 can include or use or can optionally be combined with at least some features of Aspect 1 to include or use the computer-implemented method wherein determining the difference between the first ODVH and the second ODVH includes: determining a difference between a representation of an area under a curve of the first ODVH and a representation of an area under a curve of the second ODVH.

Aspect 8 can include or use or can optionally be combined with at least some features of Aspect 7 to include or use the computer-implemented method further comprising: weighting the determined difference using a corresponding radiotherapy dose.

Aspect 9 can include or use or can optionally be combined with at least some features of Aspect 8 to include or use the computer-implemented method further comprising: normalizing the weighted determined difference.

Aspect 10 can include or use or can optionally be combined with at least some features of Aspect 1 to include or use the computer-implemented method wherein the first ODVH corresponds to a previous radiotherapy treatment fraction, and wherein the second ODVH corresponds to a current radiotherapy treatment fraction, the computer-implemented method comprising: modifying, based on the displayed output, a radiotherapy treatment plan for the current radiotherapy treatment fraction.

Aspect 11 can include or use or can optionally be combined with at least some features of Aspect 1 to include or use the computer-implemented method wherein the first ODVH corresponds to an original radiotherapy treatment plan, and wherein the second ODVH corresponds to a current radiotherapy treatment fraction, the computer-implemented method comprising: modifying, based on the displayed output, the radiotherapy treatment plan for the current radiotherapy treatment fraction.

Aspect 12 can include or use or can optionally be combined with at least some features of Aspect 1 to include or use the computer-implemented method comprising: receiving a selection, based on the displayed output, between an original radiotherapy treatment plan and a radiotherapy treatment plan generated subsequent to the original radiotherapy treatment plan.

Aspect 13 can include or use or can optionally be combined with at least some features of Aspect 12 to include or use the computer-implemented method wherein the radiotherapy treatment plan generated subsequent to the original radiotherapy treatment plan was developed during a radiotherapy treatment fraction prior to a current radiotherapy treatment fraction.

Aspect 14 can include or use subject matter (e.g., a system, apparatus, method, article, or the like) that can include or use a radiotherapy system for analyzing a radiotherapy dose-sparing opportunity for a subject, the radiotherapy system comprising: a radiation therapy device configured to deliver a dose of radiation to an anatomical region of interest; and a processor configured to: determine a representation of a first opportunity dose-volume histogram (ODVH), wherein the first ODVH is modelled based on an anatomy of the patient in a first imaging or radiotherapy treatment session; determine a representation of a second ODVH, wherein the second ODVH is modelled based on an anatomy of the patient in a second imaging or radiotherapy treatment session; determine a difference between the first ODVH and the second ODVH, wherein the difference represents the radiotherapy dose-sparing opportunity for an organ at risk (OAR); generate an output that represents the determined difference that represents an opportunity cost; and display the output to a user.

Aspect 15 can include or use or can optionally be combined with at least some features of Aspect 14 to include or use the radiotherapy system wherein the output represents a volume-specific output opportunity cost.

Aspect 17 can include or use or can optionally be combined with at least some features of Aspect 15 to include or use the radiotherapy system wherein the output represents a dose-specific opportunity cost.

Aspect 18 can include or use or can optionally be combined with at least some features of Aspect 15 to include or use the radiotherapy system wherein the output represents an aggregation of opportunity costs over multiple OARs.

Aspect 19 can include or use or can optionally be combined with at least some features of Aspect 15 to include or use the radiotherapy system, the processor further configured to: weight the output based on an importance of the OAR.

Aspect 20 can include or use or can optionally be combined with at least some features of Aspect 15 to include or use the radiotherapy system, the processor further configured to: receive an input representing a minimum dose of interest.

Aspect 21 can include or use or can optionally be combined with at least some features of Aspect 15 to include or use the radiotherapy system, wherein the processor configured to determine the difference between the first ODVH and the second ODVH is configured to: determine a difference between a representation of an area under a curve of the first ODVH and a representation of an area under a curve of the second ODVH.

Aspect 22 can include or use or can optionally be combined with at least some features of Aspect 21 to include or use the radiotherapy system, the processor further configured to: weight the determined difference using a corresponding radiotherapy dose.

Aspect 23 can include or use or can optionally be combined with at least some features of Aspect 22 to include or use the radiotherapy system, the processor further configured to: normalize the weighted determined difference.

Aspect 24 can include or use or can optionally be combined with at least some features of Aspect 15 to include or use the radiotherapy system, wherein the first ODVH corresponds to a previous radiotherapy treatment fraction, and wherein the second ODVH corresponds to a current radiotherapy treatment fraction, the processor further configured to: modify, based on the displayed output, a radiotherapy treatment plan for the current radiotherapy treatment fraction.

Aspect 25 can include or use or can optionally be combined with at least some features of Aspect 15 to include or use the radiotherapy system, wherein the first ODVH corresponds to an original radiotherapy treatment plan, and wherein the second ODVH corresponds to a current radiotherapy treatment fraction, the processor further configured to: modify, based on the displayed output, the radiotherapy treatment plan for the current radiotherapy treatment fraction.

Aspect 26 can include or use or can optionally be combined with at least some features of Aspect 15 to include or use the radiotherapy system, the processor further configured to: receive a selection, based on the displayed output, between an original radiotherapy treatment plan and a radiotherapy treatment plan generated subsequent to the original radiotherapy treatment plan.

Aspect 27 can include or use or can optionally be combined with at least some features of Aspect 26 to include or use the radiotherapy system, wherein the radiotherapy treatment plan generated subsequent to the original radiotherapy treatment plan was developed during a radiotherapy treatment fraction prior to a current radiotherapy treatment fraction.

Aspect 28 can include or use subject matter (e.g., a system, apparatus, method, article, or the like) that can include or use a treatment planning system including treatment planning circuitry, wherein the treatment planning circuitry is configured to implement any of the methods of claims 1-13.

Aspect 29 can include or use a computer-readable medium comprising instructions that, when executed, cause a processor to implement any of the methods of claims 1-13.

Opportunity analyses for plan evaluation for radiotherapy systems include:

Aspect 30 can include or use subject matter (e.g., a system, apparatus, method, article, or the like) that can include or use a computer-implemented method of evaluating a radiotherapy plan for a patient, the computer-implemented method comprising: determining, using computer processor circuitry, a representation of a first patient-specific reference dose-volume histogram (DVH) for an organ at risk (OAR); determining, using the computer processor circuitry, a representation of a comparison DVH for the OAR, wherein the comparison DVH is a second patient-specific DVH for the OAR; determining, using the computer processor circuitry, an opportunity cost that quantifies differences between the comparison DVH and the reference DVH; generating, using the computer processor circuitry, an output that represents the opportunity cost; and displaying the output to a user.

Aspect 31 can include or use or can optionally be combined with at least some features of Aspect 30 to include or use the computer-implemented method wherein the reference DVH is an ODVH.

Aspect 32 can include or use or can optionally be combined with at least some features of Aspect 30 to include or use the computer-implemented method wherein the opportunity cost is determined by an opportunity cost metric.

Aspect 33 can include or use or can optionally be combined with at least some features of Aspect 32 to include or use the computer-implemented method further comprising: normalizing, using the computer processor circuitry, the opportunity cost metric.

Aspect 34 can include or use or can optionally be combined with at least some features of Aspect 30 to include or use the computer-implemented method wherein determining, using the computer processor circuitry, the opportunity cost includes determining a mean volume difference.

Aspect 35 can include or use or can optionally be combined with at least some features of Aspect 30 to include or use the computer-implemented method wherein determining, using the computer processor circuitry, the opportunity cost includes determining a dose-weighted opportunity cost.

Aspect 36 can include or use or can optionally be combined with at least some features of Aspect 30 to include or use the computer-implemented method wherein the reference DVH and the comparison DVH are defined across a continuous dose range defined by a lower dose threshold and not by discrete points.

Aspect 37 can include or use or can optionally be combined with at least some features of Aspect 30 to include or use the computer-implemented method wherein the output represents an aggregation of opportunity costs over multiple OARs.

Aspect 38 can include or use subject matter (e.g., a system, apparatus, method, article, or the like) that can include or use a radiotherapy system for evaluating a radiotherapy plan for a patient, the radiotherapy system comprising: a radiation therapy device configured to deliver a dose of radiation to an anatomical region of interest; and a processor configured for: determining, using computer processor circuitry, a representation of a first patient-specific reference dose-volume histogram (DVH) for an organ at risk (OAR); determining, using the computer processor circuitry, a representation of a comparison DVH for the OAR, wherein the comparison DVH is a second patient-specific DVH for the OAR; determining, using the computer processor circuitry, an opportunity cost that quantifies differences between the comparison DVH and the reference DVH; generating, using the computer processor circuitry, an output that represents the opportunity cost; and displaying the output to a user.

Aspect 39 can include or use or can optionally be combined with at least some features of Aspect 38 to include or use the radiotherapy system wherein the reference DVH is an ODVH.

Aspect 40 can include or use or can optionally be combined with at least some features of Aspect 38 to include or use the radiotherapy system wherein the opportunity cost is determined by an opportunity cost metric.

Aspect 41 can include or use or can optionally be combined with at least some features of Aspect 40 to include or use the radiotherapy system further comprising: normalizing, using the computer processor circuitry, the opportunity cost metric.

Aspect 42 can include or use or can optionally be combined with at least some features of Aspect 38 to include or use the radiotherapy system wherein determining, using the computer processor circuitry, the opportunity cost includes determining a mean volume difference.

Aspect 43 can include or use or can optionally be combined with at least some features of Aspect 38 to include or use the radiotherapy system wherein determining, using the computer processor circuitry, the opportunity cost includes determining a dose-weighted opportunity cost.

Aspect 44 can include or use or can optionally be combined with at least some features of Aspect 38 to include or use the radiotherapy system wherein the reference DVH and the comparison DVH are defined across a continuous dose range defined by a lower dose threshold and not by discrete points.

Aspect 45 can include or use or can optionally be combined with at least some features of Aspect 38 to include or use the radiotherapy system wherein the output represents an aggregation of opportunity costs over multiple OARs.

Aspect 46 can include or use subject matter (e.g., a system, apparatus, method, article, or the like) that can include or use a treatment planning system including treatment planning circuitry, wherein the treatment planning circuitry is configured to implement any of the methods of claims 30-37.

Aspect 47 can include or use a computer-readable medium comprising instructions that, when executed, cause a processor to implement any of the methods of claims 30-37.

### ADDITIONAL NOTES

The above detailed description includes references to the accompanying drawings, which form a part of the detailed description. The drawings show, by way of illustration but not by way of limitation, specific embodiments in which the disclosure may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a,” “an,” “the,” and “said” are used when introducing elements of aspects of the disclosure or in the embodiments thereof, as is common in patent documents, to include one or more than one or more of the elements, independent of any other instances or usages of “at least one” or “one or more.” In this document, the term “or” is used to refer to a nonexclusive or, such that “A or B” includes “A but not B,” “B but not A,” and “A and B,” unless otherwise indicated.

In the appended claims, the terms “including” and “in which” are used as the plain-English equivalents of the respective terms “comprising” and “wherein.” Also, in the following claims, the terms “comprising,” “including,” and “having” are intended to be open-ended to mean that there may be additional elements other than the listed elements, such that elements after such a term (e.g., comprising, including, having) in a claim are still deemed to fall within the scope of that claim. Moreover, in the following claims, the terms “first,” “second,” and “third,” and so forth, are used merely as labels, and are not intended to impose numerical requirements on their objects.

Embodiments of the disclosure may be implemented with computer-executable instructions. The computer-executable instructions (e.g., software code) may be organized into one or more computer-executable components or modules. Aspects of the disclosure may be implemented with any number and organization of such components or modules. For example, aspects of the disclosure are not limited to the specific computer-executable instructions, or the specific components or modules illustrated in the figures and described herein. Other embodiments of the disclosure may include different computer-executable instructions or components having more or less functionality than illustrated and described herein.

Method examples (e.g., operations and functions) described herein may be machine or computer-implemented at least in part (e.g., implemented as software code or instructions). Some examples may include a computer-readable medium or machine-readable medium encoded with instructions operable to configure an electronic device to perform methods as described in the above examples. An implementation of such methods may include software code, such as microcode, assembly language code, a higher-level language code, or the like (e.g., “source code”). Such software code may include computer-readable instructions for performing various methods (e.g., “object” or “executable code”). The software code may form portions of computer program products. Software implementations of the embodiments described herein may be provided via an article of manufacture with the code or instructions stored thereon, or via a method of operating a communication interface to send data via a communication interface (e.g., wirelessly, over the internet, via satellite communications, and the like).

Further, the software code may be tangibly stored on one or more volatile or non-volatile computer-readable storage media during execution or at other times. These computer-readable storage media may include any mechanism that stores information in a form accessible by a machine (e.g., computing device, electronic system, and the like), such as, but are not limited to, floppy disks, hard disks, removable magnetic disks, any form of magnetic disk storage media, CD-ROMS, magnetic-optical disks, removable optical disks (e.g., compact disks and digital video disks), flash memory devices, magnetic cassettes, memory cards or sticks (e.g., secure digital cards), RAMs (e.g., CMOS RAM and the like), recordable/non-recordable media (e.g., ROMs), EPROMS, EEPROMS, or any type of media suitable for storing electronic instructions, and the like. Such computer-readable storage medium coupled to a computer system bus may be accessible by the processor and other parts of the OIS.

In an embodiment, the computer-readable storage medium may have encoded a data structure for a treatment planning, wherein the treatment plan may be adaptive. The data structure for the computer-readable storage medium may be at least one of a Digital Imaging and Communications in Medicine (DICOM) format, an extended DICOM format, an XML format, and the like. DICOM is an international communications standard that defines the format used to transfer medical image-related data between various types of medical equipment. DICOM RT refers to the communication standards that are specific to radiation therapy.

In various embodiments of the disclosure, the method of creating a component or module may be implemented in software, hardware, or a combination thereof. The methods provided by various embodiments of the present disclosure, for example, may be implemented in software by using standard programming languages such as, for example, Compute Unified Device Architecture (CUDA), C, C++, Java, Python, JavaScript and the like; and using standard machine learning/deep learning library (or API), such as tensorflow, torch and the like; and combinations thereof. As used herein, the terms “software” and “firmware” are interchangeable, and include any computer program stored in memory for execution by a computer.

A communication interface includes any mechanism that interfaces to any of a hardwired, wireless, optical, and the like, medium to communicate to another device, such as a memory bus interface, a processor bus interface, an Internet connection, a disk controller, and the like. The communication interface may be configured by providing configuration parameters and/or sending signals to prepare the communication interface to provide a data signal describing the software content. The communication interface may be accessed via one or more commands or signals sent to the communication interface.

The present disclosure also relates to a system for performing the operations herein. This system may be specially constructed for the required purposes, or it may comprise a general-purpose computer selectively activated or reconfigured by a computer program stored in the computer. The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

In view of the above, it will be seen that the several objects of the disclosure are achieved, and other beneficial results attained. Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

The above description is intended to be illustrative, and not restrictive. For example, the above-described examples (or one or more aspects thereof) may be used in combination with each other. In addition, many modifications may be made to adapt a particular situation or material to the teachings of the disclosure without departing from its scope. While the dimensions, types of materials and coatings described herein are intended to define the parameters of the disclosure, they are by no means limiting and are example embodiments. Many other embodiments will be apparent to those of skill in the art upon reviewing the above description. The scope of the disclosure should, therefore, be determined with reference to the appended claims, along with the full scope of equivalents to which such claims are entitled.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the disclosure should be determined with reference to the appended claims, along with the full scope of equivalents to which such claims are entitled. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

1-47. (canceled)

48. A computer-implemented method of analyzing a radiotherapy dose-sparing opportunity for a subject, the computer-implemented method comprising:
determining, using computer processor circuitry, a representation of a first opportunity dose-volume histogram (ODVH), wherein the first ODVH is modelled based on an anatomy of the patient in a first imaging or radiotherapy treatment session, and wherein an ODVH represents an achievable radiation dose-sparing range for an organ at risk (OAR) given a dose prescription for a target;
determining, using the computer processor circuitry, a representation of a second ODVH, wherein the second ODVH is modelled based on an anatomy of the patient in a second imaging or radiotherapy treatment session;
determining, using the computer processor circuitry, a difference between the first ODVH and the second ODVH, wherein the difference represents the radiotherapy dose-sparing opportunity for the OAR;
generating, using the computer processor circuitry, an output that represents the determined difference, wherein the determined difference represents an opportunity cost; and
displaying the output to a user.

49. The computer-implemented method of claim 48, wherein the output represents a volume-specific opportunity cost.

50. The computer-implemented method of claim 48, wherein the output represents a dose-specific opportunity cost.

51. The computer-implemented method of claim 48, wherein the output represents an aggregation of opportunity costs over multiple OARs.

52. The computer-implemented method of claim 48, further comprising:
weighting the output based on an importance of the OAR.

53. The computer-implemented method of claim 48, further comprising:
receiving an input representing a minimum dose of interest.

54. The computer-implemented method of claim 48, wherein determining the difference between the first ODVH and the second ODVH includes:
determining a difference between a representation of an area under a curve of the first ODVH and a representation of an area under a curve of the second ODVH.

55. The computer-implemented method of claim 54, further comprising:
weighting the determined difference using a corresponding radiotherapy dose.

56. The computer-implemented method of claim 55, further comprising:
normalizing the weighted determined difference.

57. The computer-implemented method of claim 48, wherein the first ODVH corresponds to a previous radiotherapy treatment fraction, and wherein the second ODVH corresponds to a current radiotherapy treatment fraction, the computer-implemented method comprising:
modifying, based on the displayed output, a radiotherapy treatment plan for the current radiotherapy treatment fraction.

58. The computer-implemented method of claim 48, wherein the first ODVH corresponds to an original radiotherapy treatment plan, and wherein the second ODVH corresponds to a current radiotherapy treatment fraction, the computer-implemented method comprising:
modifying, based on the displayed output, the radiotherapy treatment plan for the current radiotherapy treatment fraction.

59. The computer-implemented method of claim 48, comprising:
receiving a selection, based on the displayed output, between an original radiotherapy treatment plan and a radiotherapy treatment plan generated subsequent to the original radiotherapy treatment plan.

60. The computer-implemented method of claim 59, wherein the radiotherapy treatment plan generated subsequent to the original radiotherapy treatment plan was developed during a radiotherapy treatment fraction prior to a current radiotherapy treatment fraction.

61. A radiotherapy system for analyzing a radiotherapy dose-sparing opportunity for a subject, the radiotherapy system comprising:
a radiation therapy device configured to deliver a dose of radiation to an anatomical region of interest; and
a processor configured to:
determine a representation of a first opportunity dose-volume histogram (ODVH), wherein the first ODVH is modelled based on an anatomy of the patient in a first imaging or radiotherapy treatment session;
determine a representation of a second ODVH, wherein the second ODVH is modelled based on an anatomy of the patient in a second imaging or radiotherapy treatment session;
determine a difference between the first ODVH and the second ODVH, wherein the difference represents the radiotherapy dose-sparing opportunity for an organ at risk (OAR);
generate an output that represents the determined difference that represents an opportunity cost; and
display the output to a user.

62. The radiotherapy system of claim 61, wherein the output represents a volume-specific output opportunity cost.

63. The radiotherapy system of claim 62, wherein the output represents a dose-specific opportunity cost.

64. The radiotherapy system of claim 62, wherein the output represents an aggregation of opportunity costs over multiple OARs.

65. The radiotherapy system of claim 62, the processor further configured to:
weight the output based on an importance of the OAR.

66. The radiotherapy system of claim 62, the processor further configured to:
receive an input representing a minimum dose of interest.

67. The radiotherapy system of claim 62, wherein the processor configured to determine the difference between the first ODVH and the second ODVH is configured to:
determine a difference between a representation of an area under a curve of the first ODVH and a representation of an area under a curve of the second ODVH.
